SEQUENCE LISTING (12 sequences)

SEQ ID NO: 1 - DNA, 22 bp, Artificial Sequence
  Polynucleotide containing CG
tgactgtgaacgttcgagatga

SEQ ID NO: 2 - DNA, 22 bp, Artificial Sequence
  Polynucleotide containing CG
tgaccgtgaacgttcgagatga

SEQ ID NO: 3 - DNA, 23 bp, Artificial Sequence
  Polynucleotide containing CG
tcatctcgaacgttccacagtca

SEQ ID NO: 4 - DNA, 22 bp, Artificial Sequence
  Polynucleotide containing CG
tgactgtgaacgttccagatga

SEQ ID NO: 5 - DNA, 26 bp, Artificial Sequence
  Polynucleotide containing CG
tccataacgttcgcctaacgttcgtc

SEQ ID NO: 6 - DNA, 22 bp, Artificial Sequence
  Polynucleotide containing (5-bromocytosine) G
tgactgtgaangttccagatga

SEQ ID NO: 7 - DNA, 22 bp, Artificial Sequence
  Polynucleotide containing (5-bromocytosine) G
tgactgtgaangttcgagatga

SEQ ID NO: 8 - DNA, 22 bp, Artificial Sequence
  Polynucleotide containing (5-bromocytosine) G
tgactgtgaangttngagatga

SEQ ID NO: 9 - DNA, 22 bp, Artificial Sequence
  Polynucleotide not containing CG
tgactgtgaaggttagagatga

SEQ ID NO: 10 - DNA, 22 bp, Artificial Sequence
  Polynucleotide not containing CG
tcactctcttccttactcttct

SEQ ID NO: 11 - DNA, 24 bp, Artificial Sequence
  Polynucleotide containing CG
tcgtcgaacgttcgttaacgttcg

SEQ ID NO: 12 - DNA, 22 bp, Artificial Sequence
  Polynucleotide not containing CG
tgactgtgaaccttagagatga

(19) United States
(12) Patent Application Publication
(10) Pub. No.: US 2005/0059626 A1
(43) Pub. Date: Mar. 17, 2005

(54) Methods of preventing and treating viral infections using immunomodulatory polynucleotide sequences

(76) Inventors: Gary Van Nest, Martinez, CA (US); Joseph J. Eiden JR., Danville, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/898,512

(22) Filed: Jul. 22, 2004

Related U.S. Application Data
(63) Continuation of application No. 09/802,685, filed on Mar. 9, 2001, now abandoned.
(60) Provisional application No. 60/188,302, filed on Mar. 10, 2000.

(51) Int. Cl. A61K 48/00

(52) U.S. Cl. 514/44

(57) ABSTRACT

The invention provides methods of suppression, prevention, and/or treatment of infection by viruses. A polynucleotide comprising an immunostimulatory sequence (an “ISS”) is administered to an individual who is at risk of being exposed to, has been exposed to or is infected with a virus. The ISS-containing polnucleotide is administered without any antigens of the virus. Administration of the ISS-containing polynucleotide results in reduced incidence and/or severity of one or more symptoms of virus infection.

### CROSS-REFERENCE TO RELATED APPLICATIONS

This application claims the priority benefit of U.S. Provisional application 60/188,302, filed Mar. 10, 2000, which is hereby incorporated herein by reference in its entirety.

### STATEMENT OF RIGHTS TO INVENTIONS MADE UNDER FEDERALLY SPONSORED RESEARCH

Experimental work described herein was performed at the National Institutes of Health (NCI and NIAID divisions). The Government may have certain rights in this invention.

### TECHNICAL FIELD

This invention is in the field of immunostimulatory polynucleotides, more particularly to the use of immunostimulatory polynucleotides for ameliorating or preventing viral infection and symptoms of viral infection.

### BACKGROUND ART

Infections with viruses are common throughout the world. Numerous outbreaks involving viruses such as smallpox, measles, influenza, and HIV have taken their toll over the years with countless deaths. Despite much research and technological advances, viral infections remain rampant throughout the world. While some viral infections can be controlled more readily than others with commercially available drugs, many viral infections exist today that cannot be controlled and most viral infections have no cure. Drugs and/or treatment methods, such as over-the-counter cold medication or anti-retroviral drugs, have been developed to palliate the discomfort that comes from viral infections and to lessen the course of viral infections. There is no paucity in the amount of drugs and treatment methods that are specific for one virus; however, there is a lack of a treatment method that can be generally effective against multiple types of viral infections. Further, existing treatment methods, such as anti-HIV drugs or gamma globulin, are restrictive in their scope of virus specificity, namely, one treatment method cannot be used to treat multiple types of viruses. The existing treatment methods may also cause undesirable side effects, such as nausea, pain, dizziness, hair loss, autoimmune reactions or multiple drug resistance. Further, they may weaken the immune system and overall health of the individual over time with repeated administration that may result in drug toxicity.

A challenge in developing treatment methods of preventing or treating viral infections is achieving the simultaneous effect of anti-virus action without undue side effects from the composition or administration of these methods. To this end, certain DNA sequences, generally known as immunostimulatory sequences or “ISS,” emerge as a promising solution for the aforementioned difficulties.

Administration of certain DNA sequences, generally known as immunostimulatory sequences or “ISS,” induces an immune response with a Th1-type bias as indicated by secretion of Th1-associated cytokines. The Th1 subset of helper cells is responsible for classical cell-mediated functions such as activation of cytotoxic T lymphocytes (CTLs), whereas the Th2 subset functions more effectively as a helper for B-cell activation. The type of immune response to an antigen is generally influenced by the cytokines produced by the cells responding to the antigen. Differences in the cytokines secreted by Th1 and Th2 cells are believed to reflect different biological functions of these two subsets. See, for example, Romagnani (2000) Ann. Allergy Asthma Immunol. 85:9-18.

Administration of an immunostimulatory polynucleotide with an antigen results in a Th1-type immune response to the administered antigen. Roman et al. (1997) Nature Med. 3:849-854. For example, mice injected intradermally with Escherichia coli (E. coli) β-galactosidase (β-Gal) in saline or in the adjuvant alum responded by producing specific IgG1 and IgE antibodies, and CD4⁺ cells that secreted IL-4 and IL-5, but not IFN-γ, demonstrating that the T cells were predominantly of the Th2 subset. However, mice injected intradermally (or with a tyne skin scratch applicator) with plasmid DNA (in saline) encoding β-Gal and containing an ISS responded by producing IgG2a antibodies and CD4⁺ cells that secreted IFN-γ, but not IL-4 and IL-5, demonstrating that the T cells were predominantly of the Th1 subset. Moreover, specific IgE production by the plasmid DNA-injected mice was reduced 66-75%. Raz et al. (1996) Proc. Natl. Acad. Sci. USA 93:5141-5145. In general, the response to naked DNA immunization is characterized by production of IL-2, TNFα and IFN-γ by antigen-stimulated CD4⁺ T cells, which is indicative of a Th1-type response. This is particularly important in treatment of allergy and asthma as shown by the decreased IgE production. The ability of immunostimulatory polynucleotides to stimulate a Th1-type immune response has been demonstrated with bacterial antigens, viral antigens and with allergens (see, for example, WO 98/55495).

Other references describing ISS include: Krieg et al. (1989) J. Immunol. 143:2448-2451; Tokunaga et al. (1992) Microbiol. Immunol. 36:55-66; Kataoka et al. (1992) Jpn. J. Cancer Res. 83:244-247; Yamamoto et al. (1992) J. Immunol. 148:4072-4076; Mojcik et al. (1993) Clin. Immuno. and Immunopathol. 67:130-136; Branda et al. (1993) Biochem. Pharmacol. 45:2037-2043; Pisetsky et al. (1994) Life Sci. 54(2):101-107; Yamamoto et al. (1994a) Antisense Research and Development. 4:119-122; Yamamoto et al. (1994b) Jpn. J. Cancer Res. 85:775-779; Raz et al. (1994) Proc. Natl. Acad. Sci. USA 91:9519-9523; Kimura et al. (1994) J. Biochem.(Tokyo) 116:991-994; Krieg et al. (1995) Nature 374:546-549; Pisetsky et al. (1995) Ann. N.Y. Acad. Sci. 772:152-163; Pisetsky (1996a) J. Immunol. 156:421-423; Pisetsky (1996b) Immunity 5:303-310; Zhao et al. (1996) Biochem. Pharmacol. 51:173-182; Yi et al. (1996) J. Immunol. 156:558-564; Krieg (1996) Trends Microbiol. 4(2):73-76; Krieg et al. (1996) Antisense Nucleic Acid Drug Dev. 6:133-139; Klinman et al. (1996) Proc. Natl. Acad. Sci. USA. 93:2879-2883; Raz et al. (1996); Sato et al. (1996) Science 273:352-354; Stacey et al. (1996) J. Immunol. 157:2116-2122; Ballas et al. (1996) J. Immunol. 157:1840-1845; Branda et al. (1996) J. Lab. Clin. Med. 128:329-338; Sonehara et al. (1996) J. Interferon and Cytokine Res. 16:799-803; Klinman et al. (1997) J. Immunol. 158:3635-3639; Sparwasser et al. (1997) Eur. J. Immunol. 27:1671-1679; Roman et al. (1997); Carson et al. (1997) J. Exp. Med. 186:1621-1622; Chace et al. (1997) Clin. Immunol. and Immunopathol. 84:185-193; Chu et al. (1997) J. Exp. Med. 186:1623-1631; Lipford et al. (1997a) Eur. J. Immunol. 27:2340-2344; Lipford et al. (1997b) Eur. J. Immunol. 27:3420-3426; Weiner et al. (1997) Proc. Natl. Acad. Sci. USA 94:10833-10837; Macfarlane et al. (1997) Immunology 91:586-593; Schwartz et al. (1997) J. Clin. Invest. 100:68-73; Stein et al. (1997) Antisense Technology, Ch. 11 pp. 241-264, C. Lichtenstein and W. Nellen, Eds., IRL Press; Wooldridge et al. (1997) Blood 89:2994-2998; Leclerc et al. (1997) Cell. Immunol. 179:97-106; Kline et al. (1997) J. Invest. Med. 45(3):282A; Yi et al. (1998a) J. Immunol. 160:1240-1245; Yi et al. (1998b) J. Immunol. 160:4755-4761; Yi et al. (1998c) J. Immunol. 160:5898-5906; Yi et al. (1998d) J. Immunol. 161:4493-4497; Krieg (1998) Applied Antisense Oligonucleotide Technology Ch. 24, pp. 431-448, C. A. Stein and A. M. Krieg, Eds., Wiley-Liss, Inc.; Krieg et al. (1998a) Trends Microbiol. 6:23-27; Krieg et al. (1998b) J. Immunol. 161:2428-2434; Krieg et al. (1998c) Proc. Natl. Acad. Sci. USA 95:12631-12636; Spiegelberg et al. (1998) Allergy 53(45S):93-97; Horner et al. (1998) Cell Immunol. 190:77-82; Jakob et al. (1998) J. Immunol. 161:3042-3049; Redford et al. (1998) J. Immunol. 161:3930-3935; Weeratna et al. (1998) Antisense & Nucleic Acid Drug Development 8:351-356; McCluskie et al. (1998) J. Immunol. 161(9):4463-4466; Gramzinski et al. (1998) Mol. Med. 4:109-118; Liu et al. (1998) Blood 92:3730-3736; Moldoveanu et al. (1998) Vaccine 16: 1216-1224; Brazolot Milan et al. (1998) Proc. Natl. Acad. Sci. USA 95:15553-15558; Broide et al. (1998) J. Immunol. 161:7054-7062; Broide et al. (1999) Int. Arch. Allergy Immunol. 118:453-456; Kovarik et al. (1999) J. Immunol. 162:1611-1617; Spiegelberg et al. (1999) Pediatr. Pulmonol. Suppl. 18:118-121; Martin-Orozco et al. (1999) Int. Immunol. 11:1111-1118; EP 468,520; WO 96/02555; WO 97/28259; WO 98/16247; WO 98/18810; WO 98/37919; WO 98/40100; WO 98/52581; WO 98/55495; WO 98/55609 and WO 99/11275. See also Elkins et al. (1999) J. Immunol. 162:2291-2298, WO 98/52962, WO 99/33488, WO 99/33868, WO 99/51259 and WO 99/62923. See also Zimmermann et al. (1998) J. Immunol. 160:3627-3630; Krieg (1999) Trends Microbiol. 7:64-65; U.S. Pat. Nos. 5,663,153, 5,723,335, 5,849,719 and 6,174,872. See also WO 99/56755, WO 00/06588, WO 00/16804; WO 00/21556; WO 00/67023 and WO 01/12223.

There is a need for a method of treatment that can be applicable to many different types of viral infections, has efficacy in preventing or treating these viral infections and poses minimal side effects from the use of this treatment method.

All publications and patent applications cited herein are hereby incorporated by reference in their entirety.

### DISCLOSURE OF THE INVENTION

The invention provides methods of suppressing, ameliorating, and/or preventing viral infections in an individual (either before or after exposure or infection) using immunostimulatory polynucleotide sequences. Accordingly, in one aspect, the invention provides methods for preventing, palliating, ameliorating, reducing and/or eliminating one or more symptoms of viral infection. A polynucleotide comprising an immunostimulatory sequence (an “ISS”) is administered to an individual who is at risk of being exposed to a virus, has been exposed to a virus or is infected with a virus. The ISS-containing polynucleotide is administered without any viral antigens (i.e., viral antigen is not co-administered). Administration of the ISS-containing polynucleotide results in reduced incidence, recurrence and/or severity of one or more symptoms of viral infection.

In one embodiment, the invention provides methods of suppressing a virus infection in an individual at risk of being exposed to the virus which entail administering a composition comprising a polynucleotide comprising an immunostimulatory sequence (ISS) (i.e., an amount of the composition sufficient to suppress a virus infection) to the individual, wherein the ISS comprises the sequence 5′-C, G, pyrimidine, pyrimidine, C, G-3′ and wherein an antigen of the virus is not administered in conjunction with administration of the composition (i.e., antigen is not administered with the ISS-containing polynucleotide), thereby suppressing the virus infection. The individual may be at risk of being exposed to, exposed to, or infected by virus. Viral infection may be acute or chronic. In some embodiments, suppression is indicated by a reduction of titer of the virus (generally from a biological sample from the individual).

Another embodiment of the invention provides methods of preventing a symptom of virus infection in an individual which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby preventing a symptom of the virus infection. The individual may be exposed to or infected by a virus. Viral infection may be acute or chronic.

In another embodiment, the invention provides methods of reducing severity of a symptom of virus infection in an individual which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby reducing severity of a symptom of the virus infection. The individual may be exposed to or infected by a virus. Viral infection may be acute or chronic.

In another embodiment, the invention provides methods of reducing recurrence of a symptom of virus infection in an individual which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby reducing recurrence of a symptom of the virus infection. The individual may be exposed to or infected by a virus. Viral infection may be acute or chronic.

In another embodiment, the invention provides methods of reducing duration of virus infection in an individual which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby reducing duration of the virus infection. The individual may be exposed to or infected by a virus. Viral infection may be acute or chronic.

In further aspect, the invention provides methods for reducing viremia in an individual which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby reducing viremia. The individual may be exposed to or infected by a virus. Viral infection may be acute or chronic.

In a further aspect, the invention provides methods for reducing blood levels of virus antigens in an individual infected with a virus which entail administering an effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby reducing blood levels of virus antigens.

In another embodiment, the invention provides methods of delaying development of a virus infection (including delay of development of a symptom of virus infection) in an individual which entail administering effective amount of a composition comprising a polynucleotide comprising an ISS to the individual, wherein the ISS comprises the sequence 5′-C, G, pyrimidine, pyrimidine, C, G-3′ and wherein an antigen of the virus is not administered in conjunction with administration of the composition, thereby delaying development of a symptom of the virus infection.

In another aspect, the invention provides kits for use in ameliorating and/or preventing a symptom of virus infection in an individual infected with, exposed to or at risk of being exposed to a virus. The kits comprise a composition comprising a polynucleotide comprising an ISS, wherein the ISS comprises the sequence 5′-C, G, pyrimidine, pyrimidine, C, G-3′ and wherein the kit does not comprise an antigen of the virus, and wherein the kits comprise instructions for administration of the composition to an individual infected with, exposed to or at risk of being exposed to the virus.

In some embodiments of the methods and kits of the invention, the ISS comprises the sequence 5′-purine, purine, C, G, pyrimidine, pyrimidine, C, G-3′. In further embodiments of the methods and kits, the ISS comprises a sequence selected from the group consisting of AACGTTCG and GACGTTCG.

In some embodiments of the methods and kits of the invention, the ISS comprises the sequence 5′-C, G, T, T, C, G-3′. In some embodiments of the methods and kits of the invention, the ISS comprises the sequence 5′-TGACTGTGAACGTTCGAGATGA-3′ (SEQ ID NO:1).

In some embodiments of the methods and kits of the invention, the individual is a mammal. In further embodiments, the mammal is human.

In some embodiments of the invention, the ISS is administered at a site of exposure or at the site of infection.

In some embodiments of the invention, the ISS is administered systemically.

In some embodiments of the invention, administration of the ISS occurs less than about 10 days before exposure to virus.

### BRIEF DESCRIPTION OF THE DRAWINGS

FIG. 1 is a bar graph depicting lung RSV titer in rats which received intranasally: PBS (first bar); ISS three days before viral infection (second bar) non-ISS control sequence three days before viral infection (third bar); ISS 30 minutes before viral infection (fourth bar); non-ISS control sequence 30 minutes before viral infection.

FIG. 2A-2D are graphs depicting effects of administration of ISS and control reagents to STC mice on HBV viral titer. Results shown are blood HBV DNA titer (in copies per milliliter) over time (in days). FIG. 2A depicts results for STC mice injected with ISS at day 0, 7, and 14 (week 0, 1 and 2); FIG. 2B depicts results for STC mice injected with ISS at day 14 (week 2) only; FIG. 2C depicts results for STC mice injected with 100 ng of murine IL-12 on days 12, 13 and 14; and FIG. 2D depicts results for STC mice injected with phosphate buffered saline (PBS) on days 0, 7 and 14. Error bars indicate±one standard deviation (SD).

FIG. 3 is a graph depicting effects of administration of ISS and control reagents to STC mice on hepatitis B surface antigen (HBsAg) levels. Results are shown as percent of value at day −1 over time (in days). Open squares indicate results for STC mice injected with ISS at day 0, 7, and 14 (week 0, 1 and 2); closed diamonds indicate results for STC mice injected with ISS at day 14 (week 2) only; closed square indicate results for STC mice injected with 100 ng of murine IL-12 on days 12, 13 and 14; and open diamonds indicate results for STC mice injected with phosphate buffered saline on days 0, 7 and 14.

FIG. 4 summarizes results of ISS treatment of mice infected with HSV2. The graph depicts animal survival following a lethal challenge dose of HSV2 and subsequent treatment regimens. Animals that received an ISS treatment demonstrated improved survival as compared to animals that received non-ISS oligonucleotide treatments, PBS or no treatment.

FIG. 5 summarizes results of ISS treatment of guinea pigs infected with HSV2. The graphs depict cumulative mean herpetic lesions over the observation period in groups of animals receiving a single ISS treatment (“ISS 1”), receiving a total of three ISS treatments (“ISS 3”) or receiving PBS alone (“sham”).

FIG. 6 summarizes results of ISS treatment of guinea pigs infected with HSV2. The graph depicts cumulative mean herpetic lesions over the observation period in groups of animals receiving a single ISS treatment, a single non-ISS oligonucleotide treatment, 21 acyclovir treatments or no treatment.

FIG. 7 is a graphical depiction of the average number of genomic equivalents per shedding event from herpetic lesions in guinea pigs.

FIG. 8 is a bar graph depicting results of ISS treatment in a canine model of papillomavirus for time of wart regression.

FIG. 9 are graphs depicting results of ISS treatment of papillomavirus in a rabbit model. The data is expressed as geometric mean diameter (GMD) over time after inoculation. Closed circles indicate Group A animals, open circles indicate Group B animals, and closed triangles indicate Group C animals. FIG. 9(A) depicts GMD for the left side, high CRPV dose lesions. FIG. 9(B) depicts GMD for the left side, low CRPV dose lesions. FIG. 9(C) depicts average GMD for the right side, high CRPV dose lesions. FIG. 9(D) depicts average GMD for the right side, low CRPV dose lesions.

FIG. 10 is a graph depicting results of ISS treatment of rabbit papillomavirus. The data is expressed as geometric mean diameter (GMD) over time after inoculation. Closed circles indicate ISS treated papilloma sites, open circles indicate untreated papilloma sites animals, and downward arrows indicate timing of ISS treatments.

### MODES FOR CARRYING OUT THE INVENTION

We have discovered methods of preventing and/or treating viral infections using immunomodulatory polynucleotides that induce anti-viral immune responses and promote anti-viral effects. A polynucleotide comprising an immunostimulatory sequence (an “ISS”) is administered to an individual at risk of being exposed to, exposed to or infected with a virus. Administration of ISS-containing polynucleotide without co-administration of a viral antigen results in prevention and/or reduction of severity of one or more symptoms of viral infection. These methods are applicable to a number of different viruses.

The invention also relates to kits for ameliorating and/or preventing a symptom of virus infection in exposed individuals. The kits, which do not contain a viral antigen, comprise a polynucleotide comprising an ISS and instructions describing the administration of an ISS-containing polynucleotide to an individual for the intended treatment.

We have used several art-accepted models of viral infection including models of hepatitis virus, papillomavirus, respiratory virus and herpesvirus. We have shown that administration of ISS-containing polynucleotide is effective at reducing viral titers.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); The Immunoassay Handbook (David Wild, ed., Stockton Press NY, 1994); and Methods of Immunological Analysis (R. Masseyeff, W. H. Albert, and N. A. Staines, eds., Weinheim: VCH Verlags gesellschaft mbH, 1993).

Definitions

The term “virus” refers to an infectious, replicating, submicroscopic agent which can be characterized by properties including, but not limited to, virion size, shape, other related morphology (capsid symmetry, presence or absence of envelope, etc.), physical properties (virion molecular mass, virion sedimentation coefficient, thermal stability, pH stability, etc.), genome (type of nucleic acid DNA or RNA, size of genome, single stranded or double-stranded, linear or circular, positive-sense or negative-sense or ambisense, segmentation, etc.), proteins (number of proteins encoded, number of open reading frames, glycosylation of proteins, etc.), lipid content, carbohydrate content, antigenic properties, and biological properties (natural host range, mode of transmission, geographic distribution, vector relationships, tissue or cellular tropisms, etc.). The term “virus” encompasses both pathogenic and non-pathogenic viruses.

The term “pathogenic viruses” refers to viruses which cause clinical disease in the host. Examples of pathogenic viruses include, but are not limited to, hepatitis B virus, human immunodeficiency virus (HIV), respiratory viruses (such as RSV), papillomaviruses and measles virus. “Non-pathogenic viruses” refer to viruses which do not cause clinical disease in the host. Examples of non-pathogenic viruses include, but are not limited to, hepatitis G virus, adeno-associated virus (AAV), and transfusion-transmission virus (TTV).

“Exposure” to a virus denotes encounter with virus which allows infection, such as, for example, upon contact with an infected individual, contact with virus contaminated surfaces or contact, particularly percutaneous contact, with bodily fluids containing virus.

An individual is “seronegative” for a virus if antibodies specific to the virus cannot be detected in blood or serum samples from the individual using methods standard in the art, such as ELISA. Conversely, an individual is “seropositive” for a virus if antibodies specific for the virus can be detected in blood or serum samples from the individual using methods standard in the art, such as ELISA. An individual is said to “seroconvert” for a virus when antibodies to the virus can be detected in blood or serum from an individual who was previously seronegative.

An individual who is “at risk of being exposed” to a virus is an individual who may encounter the virus such that the virus infects the individual (i.e., virus enters cells and replicates). In the context of viruses which causes acute infection and resolution of infection and symptoms, the individual may or may not have previously been exposed to virus, but it is understood that, at the time of at least one administration of ISS-containing polynucleotide, the individual is symptom-free and has not been exposed to virus within about 5 days of administration of ISS. Because many viruses, including pathogenic viruses, are ubiquitous, generally any individual is at risk for exposure to the virus. In some contexts, an individual is determined to be “at risk” because exposure to the virus has higher probability of leading to infection (such as with immunocompromised, elderly and/or very young children and infants) which can further result in serious symptoms, conditions, and/or complications. In some settings, including, but not limited to, institutions such as hospitals, schools, day care facilities, dialysis facilities, military facilities, nursing homes and convalescent homes, an individual is determined to be “at risk” because of time spent in close proximity to others who may be infected. In the context of some viruses, an individual at risk of being exposed to a virus is any individual who is seronegative for the virus (e.g., herpes simplex viruses types 1 and 2). In other contexts, an individual at risk of being exposed to the virus is an individual who is engaging in one or more high risk behaviors (e.g., sexual relations without the use of barrier prophylactics in the case of HPV and HSV2).

“Viral infection” used herein denotes infection of individual by one or more virus(es) that may belong to different species, genera, subfamilies, families, or orders, according to the International Committee on Taxonomy of Viruses (ICTV) guidelines. “Viral infection” includes chronic or acute viral infection.

As well known in the art, an “acute infection” is, generally, an infection with a rapid onset and/or short duration. An acute infection is not a chronic infection.

As well known in the art, a “chronic infection” is an infection which is generally, long and/or drawn out in duration. A chronic infection is not an acute infection.

“Suppressing” viral infection indicates any aspect of viral infection, such as viral replication, time course of infection, amount (titer) of virus, lesions, and/or one or more symptoms is curtailed, inhibited, or reduced (in terms of severity and/or duration) in an individual or population of individuals treated with an ISS-containing polynucleotide in accordance with the invention as compared to an aspect of viral infection in an individual or population of individuals not treated in accordance with the invention. Reduction in viral titer includes, but is not limited to, elimination of the virus from an infected site or individual. Viral infection can be assessed by any means known in the art, including, but not limited to, measurement of virus particles, viral nucleic acid or viral antigens, detection of symptoms and detection and/or measurement of anti-virus antibodies. Anti-virus antibodies are widely used to detect and monitor viral infection and generally are commercially available. In addition, viral infection can be assessed by other means known in the art including, but not limited to, PCR, in situ hybridization with virus specific probes, infectious center assays, plaque assays, etc.

“Palliating” a disease or one or more symptoms of a disease or infection means lessening the extent and/or time course of undesirable clinical manifestations of a disease state or infection in an individual or population of individuals treated with an ISS in accordance with the invention.

As used herein, “delaying” development of a viral infection or a symptom of a viral infection means to defer, hinder, slow, retard, stabilize, and/or postpone development of the disease or symptom when compared to not using the method(s) of the invention. This delay can be of varying lengths of time, depending on the history of the disease and/or individual being treated. As is evident to one skilled in the art, a sufficient or significant delay can, in effect, encompass prevention, in that the individual does not develop the disease.

“Symptoms of viral infection” used herein refers to any aspect of virus infection, such as a physical symptom (e.g., jaundice, fatigue, malaise, vomiting, abdominal pain, fever, lesions, warts, epidermal abnormalities, sore throat, inflammation of mucosa, fever, body aches, coughing, wheezing, sneezing, nasal discharge, chest pain), a virus-associated laboratory finding (e.g., enzyme levels such as ALT, AST, and/or LDH, elevated bilirubin, histological analysis of biopsies, MRI, CT, X-rays, or evidence of metastasis), viral replication, viral shedding, or amount (titer) of virus. Detection of virus, viral infection, or viral shedding can be assessed by any means known in the art, including, but not limited to, PCR of biological fluids, cells, tissues, in situ hybridization with virus specific probes, measurement of virus by limiting dilution assays, infectious center assays, histological examination of biological samples, and cell culturing of virus isolated from infected individuals.

“Reducing severity of a symptom” or “ameliorating a symptom” of viral infection means a lessening, improvement, or amelioration of one or more symptoms of viral infection as compared to not administering an ISS-containing polynucleotide. “Reducing severity” also includes shortening or reduction in duration of a symptom. For example, in infections with influenza virus and other respiratory viruses, these symptoms are well-known in the art and include, but are not limited to, inflammation of mucosa, fever, body aches, coughing, wheezing, sneezing, nasal discharge and chest pain. In another example with hepatitis B and C, the term “symptom of HBV or HCV” refers to acute and chronic hepatitis B and C symptoms that are well known in the art and include physical symptoms such as jaundice, abdominal pain, fatigue, malaise, nausea, and vomiting, as well as clinical/laboratory findings associated with hepatitis, such as elevated liver enzyme levels (e.g., ALT, AST, and/or LDH), elevated bilirubin, HBV and/or HCV viremia, portal hypertension, cirrhosis and other symptoms recognized in the art. In another example of viral infection with herpesvirus (or other members of alphaherpesvirinae), symptoms include, but are not limited to, cutaneous or mucosal lesions and viral shedding. In another example of viral infection with papillomavirus (or other members of the papillomavirinae), symptoms include, but are not limited to, the clinical presentation of warts, condyloma and papilloma, all of which can be collectively referred to as “lesions” and other symptoms associated with warts, condyloma, papilloma and lesions which can include, but is not limited to, hoarseness of voice, breathing difficulties, pain and discomfort. In another example of viral infection, infection with arenaviruses such as lymphocytic choriomeningitis virus (LCMV), Lassa virus, and Sabia virus, symptoms include, but are not limited to, nausea, myalgia, dizziness, vomiting, diarrhea, prostration, headache, photophobia, fever, malaise, leukopenia, thrombocytopenia, hemorrhaging of the skin and internal organs and focal necrosis of organs such as the liver.

“Suppressing a symptom of virus infection” refers to any one or more symptoms associated with viral infection, described above, which are curtailed, inhibited, or reduced (in terms of severity and/or duration) in an individual or a population of individuals treated with an ISS in accordance with the invention as compared to an aspect of viral infection in an individual or a population of individuals not treated in accordance with the invention. Viral infection can be assessed by any means known in the art, including, but not limited to, measurement of virus, detection and/or quantitation of symptoms, laboratory testing of biological fluids, and appearance of well-characterized viral lesions.

“Preventing a symptom of infection” by a virus means that the symptom does not appear after exposure to the virus. Examples of symptoms of viral infections have been described above.

“Reducing duration of viral infection” means the length of time of viral infection (usually indicated by symptoms) is reduced, or shortened, as compared to not administering an ISS-containing polynucleotide.

“Reducing recurrence” refers to a reduction in frequency, severity and/or quantity of one or more recurrent viral symptoms in an infected individual or a population of infected individuals. When applied to a population of individuals, “reducing recurrence” means a reduction in the mean or median frequency, severity, quantity and/or duration of recurrent viral symptoms.

The term “infected individual”, as used herein, refers to an individual who has been infected by a virus.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“Viral titer” is a term well known in the art and indicates the amount of virus in a given biological sample. “Viremia” is a term well-known in the art as the presence of virus in the blood stream and/or viral titer in a blood or serum sample. Amount of virus are indicated by various measurements, including, but not limited to, amount of viral nucleic acid; presence of viral particles; replicating units (RU); plaque forming units (PFU). Generally, for fluid samples such as blood and urine, amount of virus is determined per unit fluid, such as milliliters. For solid samples such as tissue samples, amount of virus is determined per weight unit, such as grams. Methods for determining amount of virus are known in the art and described herein.

An “individual” is a vertebrate, preferably a mammal, more preferably a human. Mammals include, but are not limited to, humans, farm animals, sport animals, rodents, primates and certain pets. Vertebrates also include, but are not limited to, birds (i.e., avian individuals) and reptiles (i.e., reptilian individuals).

The term “ISS” as used herein refers to polynucleotide sequences that effect a measurable immune response as measured in vitro, in vivo and/or ex vivo. Examples of measurable immune responses include, but are not limited to, antigen-specific antibody production, secretion of cytokines, activation or expansion of lymphocyte populations such as NK cells, CD4⁺ T lymphocytes, CD8⁺ T lymphocytes, B lymphocytes, and the like. Preferably, the ISS sequences preferentially activate a Th1-type response. A polynucleotide for use in methods of the invention contains at least one ISS.

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The polynucleotide can be linearly or circularly configured, or the polynucleotide can contain both linear and circular segments.

“Adjuvant” refers to a substance which, when added to an immunogenic agent such as antigen, nonspecifically enhances or potentiates an immune response to the agent in the recipient host upon exposure to the mixture.

An “effective amount” or a “sufficient amount” of a substance is an amount sufficient to effect beneficial or desired results, including clinical results. An effective amount can be administered in one or more administrations. A “therapeutically effective amount” is an amount to effect beneficial clinical results, including, but not limited to, alleviation of one or more symptoms associated with viral infection as well as prevention of disease (e.g., prevention of one or more symptoms of infection).

A microcarrier is considered “biodegradable” if it is degradable or erodable under normal mammalian physiological conditions. Generally, a microcarrier is considered biodegradable if it is degraded (i.e., loses at least 5% of its mass and/or average polymer length) after a 72 hour incubation at 37° C. in normal human serum. Conversely, a microcarrier is considered “nonbiodegradable” if it is not degraded or eroded under normal mammalian physiological conditions. Generally, a microcarrier is considered nonbiodegradable if it not degraded (i.e., loses less than 5% of its mass and/or average polymer length) after at 72 hour incubation at 37° C. in normal human serum.

The term “immunostimulatory sequence-microcarrier complex” or “ISS-MC complex” refers to a complex of an ISS-containing polynucleotide and a microcarrier. The components of the complex may be covalently or non-covalently linked. Non-covalent linkages may be mediated by any non-covalent bonding force, including by hydrophobic interaction, ionic (electrostatic) bonding, hydrogen bonds and/or van der Waals attractions. In the case of hydrophobic linkages, the linkage is generally via a hydrophobic moiety (e.g., cholesterol) covalently linked to the ISS.

As used herein, the term “comprising” and its cognates are used in their inclusive sense; that is, equivalent to the term “including” and its corresponding cognates.

As used herein, the singular form “a”, “an”, and “the” includes plural references unless indicated otherwise. For example, “a” symptom of viral infection includes one or more additional symptoms.

Methods of Invention

The invention provides methods of ameliorating and/or preventing one or more symptoms of viral infection as well as methods of suppressing and/or preventing infection by viruses which entail administering an ISS-containing polynucleotide (used interchangeably herein with “ISS”) to an individual without administering a viral antigen. An ISS-containing composition which does not include a viral antigen is administered to an individual at risk of exposure to, exposed to, infected with, and/or exhibiting symptoms of infection by a virus. Individuals receiving ISS are preferably mammal, more preferably human. In accordance with the invention, ISS is administered without any viral antigens. Virus antigen is not administered to the individual in conjunction with administration of an ISS (i.e., is not administered in a separate administration at or about the time of administration of the ISS).

The virus may be any virus including pathogenic and non-pathogenic virus. Using the most current report of “The Classification and Nomenclature of Viruses” guidelines set forth in 1995 by the International Committee on Taxonomy of Viruses (ICTV), individuals, preferably humans, that are infected may be infected with one or more species of virus(es). Further, the viruses can be different species or from different genera, different subfamilies, different families, or different orders. The mode of transmission may include, but is not limited to, airborne transmissions, aerosolized transmission, sexual contact, surface-to-surface contact, secondary vectors (e.g., mosquitoes, flies, worms, parasites, etc.), ingestion of contaminated food or liquids, blood transfusion, introduction of virus into the individual through accidents such as laboratory or clinical error (e.g., cut during necropsy, injection of virus intended for cell culture or animal testing purposes), bites from infected individuals and biological fluid intake from infected individuals. Examples of virus include, but are not limited to, respiratory virus (including RSV), hepatitis virus (including hepatitis B virus (HBV) and hepatitis C virus (HCV)), herpes virus (including herpes simplex virus 1 (HSV1), herpes simplex virus 2 (HSV2) and varacella zoster virus (VZV)), papillomavirus (including human papillomavirus (HPV)) and human immunodeficiency virus (HIV).

In some embodiments, the individual is at risk of being exposed to virus. Determination of an at risk individual is based on one or more factors that are associated with disease development, mode of transmission, opportunity for viral infection, accessibility of vectors and/or virus to the at risk individual and are generally known by, or can be assessed by, a skilled clinician. At risk individuals may be especially suitable candidates to receive ISS-containing polynucleotides, as these individuals are generally considered to be particularly susceptible to developing symptoms of infection, which could also further lead to other complications. For example, in the context of RSV infection, age groups of about 2 years or less, the elderly and those with immunocompromised systems would be considered at risk. In another example, in the context of sexually transmitted viral infections such as HIV, herpesvirus, papillomavirus and hepatitis, individuals considered to be at risk would include, but is not limited to, immunocompromised individuals and individuals with opportunity for exposure or by association with those individuals with opportunities for exposure (e.g., spouses, partners, prostitutes, etc.)

In other embodiments, the individual is, or has been, exposed to and/or infected by virus. Exposure to virus is generally indicated by sufficient contact with an infected individual or infected location. Exposure can also be indicated by development of one or more symptoms associated with viral infection. Infection by virus may be indicated by any of the above, as well as detection of virus or anti-virus antibodies (i.e., the individual becomes seropositive) in a biological sample from the individual. Infection may be acute or chronic.

In further embodiments, the individual is, or has been, exposed to and infected by virus(es), and has not yet developed any symptoms associated with the viral infection. The symptoms will vary depending on which type or types of virus(es) have infected the individual. Identification of these symptoms is readily done by a skilled clinician. For example, symptoms of papillomavirus infection may be genital lesions or warts.

ISS

The methods of this invention entail administering a polynucleotide comprising an ISS (or a composition comprising such a polynucleotide). In accordance with the present invention, the immunomodulatory polynucleotide contains at least one ISS, and can contain multiple ISSs. The ISSs can be adjacent within the polynucleotide, or they can be separated by additional nucleotide bases within the polynucleotide. Alternately, multiple ISSs may be delivered as individual polynucleotides.

ISS have been described in the art and may be readily identified using standard assays which indicate various aspects of the immune response, such as cytokine secretion, antibody production, NK cell activation and T cell proliferation. See, e.g., WO 97/28259; WO 98/16247; WO 99/11275; Krieg et al. (1995); Yamamoto et al. (1992); Ballas et al. (1996); Klinman et al. (1997); Sato et al. (1996); Pisetsky (1996a); Shimada et al. (1986) Jpn. J. Cancer Res. 77:808-816; Cowdery et al. (1996) J. Immunol. 156:4570-4575; Roman et al. (1997); and Lipford et al. (1997a).

The ISS can be of any length greater than 6 bases or base pairs and generally comprises the sequence 5′-cytosine, guanine-3′, preferably greater than 15 bases or base pairs, more preferably greater than 20 bases or base pairs in length. As is well-known in the art, the cytosine of the 5′-cytosine, guanine-3′ sequence is unmethylated. An ISS may also comprise the sequence 5′-purine, purine, C, G, pyrimidine, pyrimidine, C, G-3′. An ISS may also comprise the sequence 5′-purine, purine, C, G, pyrimidine, pyrimidine, C, C-3′. As indicated in polynucleotide sequences below, an ISS may comprise (i.e., contain one or more of) the sequence 5′-T, C, G-3′. In some embodiments, an ISS may comprise the sequence 5′-C, G, pyrimidine, pyrimidine, C, G-3′ (such as 5′-CGTTCG-3′). In some embodiments, an ISS may comprise the sequence 5′-C, G, pyrimidine, pyrimidine, C, G, purine, purine-3′. In some embodiments, an ISS comprises the sequence 5′-purine, purine, C, G, pyrimidine, pyrimidine-3′ (such as 5′-AACGTT-3′).

In some embodiments, an ISS may comprise the sequence 5′-purine, T, C, G, pyrimidine, pyrimidine-3′.

In some embodiments, an ISS-containing polynucleotide is less than about any of the following lengths (in bases or base pairs): 10,000; 5,000; 2500; 2000; 1500; 1250; 1000; 750; 500; 300; 250; 200; 175; 150; 125; 100; 75; 50; 25; 10. In some embodiments, an ISS-containing polynucleotide is greater than about any of the following lengths (in bases or base pairs): 8; 10; 15; 20; 25; 30; 40; 50; 60; 75; 100; 125; 150; 175; 200; 250; 300; 350; 400; 500; 750; 1000; 2000; 5000; 7500; 10000; 20000; 50000. Alternately, the ISS can be any of a range of sizes having an upper limit of 10,000; 5,000; 2500; 2000; 1500; 1250; 1000; 750; 500; 300; 250; 200; 175; 150; 125; 100; 75; 50; 25; or 10 and an independently selected lower limit of 8; 10; 15; 20; 25; 30; 40; 50; 60; 75; 100; 125; 150; 175; 200; 250; 300; 350; 400; 500; 750; 1000; 2000; 5000; 7500, wherein the lower limit is less than the upper limit.

In some embodiments, the ISS comprises any of the following sequences:

GACGCTCC; GACGTCCC; GACGTTCC; GACGCCCC; AGCGTTCC;
AGCGCTCC; AGCGTCCC; AGCGCCCC; AACGTCCC; AACGCCCC;
AACGTTCC; AACGCTCC; GGCGTTCC; GGCGCTCC; GGCGTCCC;
GGCGCCCC; GACGCTCG; GACGTCCG; GACGCCCG; GACGTTCG;
AGCGCTCG; AGCGTTCG; AGCGTCCG; AGCGCCCG; AACGTCCG;
AACGCCCG; AACGTTCG; AACGCTCG; GGCGTTCG; GGCGCTCG;
GGCGTCCG; GGCGCCCG.

In some embodiments, the immunomodulatory polynucleotide comprises the sequence 5′-TGACTGTGAACGTTCGAGATGA-3′ (SEQ ID NO:1).

In some embodiments, the ISS comprises any of the following sequences:

GACGCU; GACGUC; GACGUU; GACGUT; GACGTU; AGCGUU;
AGCGCU; AGCGUC; AGCGUT; AGCGTU; AACGUC; AACGUU;
AACGCU; AACGUT; AACGTU; GGCGUU; GGCGCU; GGCGUC;
GGCGUT; GGCGTU.

In some embodiments, the ISS comprises any of the following sequences:

GABGCTCC; GABGTCCC; GABGTTCC; GABGCCCC; AGBGTTCC;
AGBGCTCC; AGBGTCCC; AGBGCCCC; AABGTCCC; AABGCCCC;
AABGTTCC; AABGCTCC; GGBGTTCC; GGBGCTCC; GGBGTCCC;
GGBGCCCC; GABGCTCG; GABGTCCG; GABGCCCG; GABGTTCG;
AGBGCTCG; AGBGTTCG; AGBGTCCG; AGBGCCCG; AABGTCCG;
AABGCCCG; AABGTTCG; AABGCTCG; GGBGTTCG; GGBGCTCG;
GGBGTCCG; GGBGCCCG; GABGCTBG; GABGTCBG; GABGCCBG;
GABGTTBG; AGBGCTBG; AGBGTTBG; AGBGTCBG; AGBGCCBG;
AABGTCBG; AABGCCBG; AABGTTBG; AABGCTBG; GGBGTTBG;
GGBGCTBG; GGBGTCBG; GGBGCCBG, where B is
5-bromocytosine.

In some embodiments, the ISS comprises any of the following sequences:

GABGCUCC; GABGUCCC; GABGUTCC; GABGTUCC; GABGUUCC;
AGBGUUCC; AGBGTUCC; AGBGUTCC; AGBGCUCC; AGBGUCCC;
AABGUCCC; AABGUUCC; AABGUTCC; AABGTUCC; AABGCUCC;
GGBGUUCC; GGBGUTCC; GGBGTUCC; GGBGCUCC; GGBGUCCC;
GABGCUCG; GABGUCCG; GABGUUCG; GABGUTCG; GABGTUCG;
AGBGCUCG; AGBGUUCG; AGBGUTCG; AGBGTUCG; AGBGUCCG;
AABGUCCG; AABGUUCG; AABGUTCG; AABGTUCG; AABGCUCG;
GGBGUUCG; GGBGUTCG; GGBGTUCG; GGBGCUCG; GGBGUCCG;
GABGCUBG; GABGUCBG; GABGUUBG; GABGUTBG; GABGTUBG;
AGBGCUBG; AGBGUUBG; AGBGUCBG; AGBGUTBG; AGBGTUBG;
AABGUCBG; AABGUUBG; AABGUTBG; AABGTUBG; AABGCUBG;
GGBGUUBG; GGBGUTBG; GGBGTUBG; GGBGCUBG; GGBGUCBG,

where B is 5-bromocytosine.

In other embodiments, the ISS comprises any of the sequences:

| | |
|---|---|
| 5′-TGACCGTGAACGTTCGAGATGA-3′; | (SEQ ID NO: 2) |
| 5′-TCATCTCGAACGTTCCACAGTCA-3′; | (SEQ ID NO: 3) |
| 5′-TGACTGTGAACGTTCCAGATGA-3′; | (SEQ ID NO: 4) |
| 5′-TCCATAACGTTCGCCTAACGTTCGTC-3′; | (SEQ ID NO: 5) |
| 5′-TGACTGTGAABGTTCCAGATGA-3′, | (SEQ ID NO: 6) |

where B is 5-bromocytosine;
5′-TGACTGTGAABGTTCGAGATGA-3′ (SEQ ID NO:7), where B is 5-bromocytosine and
5′-TGACTGTGAABGTTBGAGATGA-3′ (SEQ ID NO:8), where B is 5-bromocytosine.

In some embodiments, the immunomodulatory polynucleotide comprises the sequence 5′-TCGTCGAACGTTCGTTAACGTTCG-3′ (SEQ ID NO:11).

An ISS and/or ISS-containing polynucleotide may contain modifications. Modifications of ISS include any known in the art, but are not limited to, modifications of the 3′-OH or 5′-OH group, modifications of the nucleotide base, modifications of the sugar component, and modifications of the phosphate group. Various such modifications are described below.

An ISS may be single stranded or double stranded DNA, as well as single or double-stranded RNA or other modified polynucleotides. An ISS may or may not include one or more palindromic regions, which may be present in the motifs described above or may extend beyond the motif. An ISS may comprise additional flanking sequences, some of which are described herein. An ISS may contain naturally-occurring or modified, non-naturally occurring bases, and may contain modified sugar, phosphate, and/or termini. For example, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. Preferably, oligonucleotides of the present invention comprise phosphorothioate backbones. Sugar modifications known in the field, such as 2′-alkoxy-RNA analogs, 2′-amino-RNA analogs and 2′-alkoxy- or amino-RNA/DNA chimeras and others described herein, may also be made and combined with any phosphate modification. Examples of base modifications include, but are not limited to, addition of an electron-withdrawing moiety to C-5 and/or C-6 of a cytosine of the ISS (e.g., 5-bromocytosine, 5-chlorocytosine, 5-fluorocytosine, 5-iodocytosine).

The ISS can be synthesized using techniques and nucleic acid synthesis equipment which are well known in the art including, but not limited to, enzymatic methods, chemical methods, and the degradation of larger oligonucleotide sequences. See, for example, Ausubel et al. (1987); and Sambrook et al. (1989). When assembled enzymatically, the individual units can be ligated, for example, with a ligase such as T4 DNA or RNA ligase. U.S. Pat. No. 5,124,246. Oligonucleotide degradation can be accomplished through the exposure of an oligonucleotide to a nuclease, as exemplified in U.S. Pat. No. 4,650,675.

The ISS can also be isolated using conventional polynucleotide isolation procedures. Such procedures include, but are not limited to, hybridization of probes to genomic or cDNA libraries and synthesis of particular native sequences by the polymerase chain reaction.

Circular ISS can be isolated, synthesized through recombinant methods, or chemically synthesized. Where the circular ISS is obtained through isolation or through recombinant methods, the ISS will preferably be a plasmid. The chemical synthesis of smaller circular oligonucleotides can be performed using any method described in the literature. See, for instance, Gao et al. (1995) Nucleic Acids Res. 23:2025-2029; and Wang et al. (1994) Nucleic Acids Res. 22:2326-2333.

The techniques for making oligonucleotides and modified oligonucleotides are known in the art. Naturally occurring DNA or RNA, containing phosphodiester linkages, is generally synthesized by sequentially coupling the appropriate nucleoside phosphoramidite to the 5′-hydroxy group of the growing oligonucleotide attached to a solid support at the 3′-end, followed by oxidation of the intermediate phosphite triester to a phosphate triester. Once the desired oligonucleotide sequence has been synthesized, the oligonucleotide is removed from the support, the phosphate triester groups are deprotected to phosphate diesters and the nucleoside bases are deprotected using aqueous ammonia or other bases. See, for example, Beaucage (1993) “Oligodeoxyribonucleotide Synthesis” in Protocols for Oligonucleotides and Analogs, Synthesis and Properties (Agrawal, ed.) Humana Press, Totowa, N.J.; Warner et al. (1984) DNA 3:401 and U.S. Pat. No. 4,458,066.

The ISS can also contain phosphate-modified oligonucleotides. Synthesis of polynucleotides containing modified phosphate linkages or non-phosphate linkages is also know in the art. For a review, see Matteucci (1997) “Oligonucleotide Analogs: an Overview” in Oligonucleotides as Therapeutic Agents, (D. J. Chadwick and G. Cardew, ed.) John Wiley and Sons, New York, N.Y. The phosphorous derivative (or modified phosphate group) which can be attached to the sugar or sugar analog moiety in the oligonucleotides of the present invention can be a monophosphate, diphosphate, triphosphate, alkylphosphonate, phosphorothioate, phosphorodithioate or the like. The preparation of the above-noted phosphate analogs, and their incorporation into nucleotides, modified nucleotides and oligonucleotides, per se, is also known and need not be described here in detail. Peyrottes et al. (1996) Nucleic Acids Res. 24:1841-1848; Chaturvedi et al. (1996) Nucleic Acids Res. 24:2318-2323; and Schultz et al. (1996) Nucleic Acids Res. 24:2966-2973. For example, synthesis of phosphorothioate oligonucleotides is similar to that described above for naturally occurring oligonucleotides except that the oxidation step is replaced by a sulfurization step (Zon (1993) “Oligonucleoside Phosphorothioates” in Protocols for Oligonucleotides and Analogs, Synthesis and Properties (Agrawal, ed.) Humana Press, pp. 165-190). Similarly the synthesis of other phosphate analogs, such as phosphotriester (Miller et al. (1971) JACS 93:6657-6665), non-bridging phosphoramidates (Jager et al. (1988) Biochem. 27:7247-7246), N3′ to P5′ phosphoramidates (Nelson et al. (1997) JOC 62:7278-7287) and phosphorodithioates (U.S. Pat. No. 5,453,496) has also been described. Other non-phosphorous based modified oligonucleotides can also be used (Stirchak et al. (1989) Nucleic Acids Res. 17:6129-6141). Oligonucleotides with phosphorothioate backbones can be more immunogenic than those with phosphodiester backbones and appear to be more resistant to degradation after injection into the host. Braun et al. (1988) J. Immunol. 141:2084-2089; and Latimer et al. (1995) Mol. Immunol. 32:1057-1064.

ISS-containing polynucleotides used in the invention can comprise ribonucleotides (containing ribose as the only or principal sugar component), deoxyribonucleotides (containing deoxyribose as the principal sugar component), or, as is known in the art, modified sugars or sugar analogs can be incorporated in the ISS. Thus, in addition to ribose and deoxyribose, the sugar moiety can be pentose, deoxypentose, hexose, deoxyhexose, glucose, arabinose, xylose, lyxose, and a sugar “analog” cyclopentyl group. The sugar can be in pyranosyl or in a furanosyl form. In the ISS, the sugar moiety is preferably the furanoside of ribose, deoxyribose, arabinose or 2′-0-alkylribose, and the sugar can be attached to the respective heterocyclic bases either in α or β anomeric configuration. Sugar modifications include, but are not limited to, 2′-alkoxy-RNA analogs, 2′-amino-RNA analogs and 2′-alkoxy- or amino-RNA/DNA chimeras. The preparation of these sugars or sugar analogs and the respective “nucleosides” wherein such sugars or analogs are attached to a heterocyclic base (nucleic acid base) per se is known, and need not be described here, except to the extent such preparation can pertain to any specific example. Sugar modifications may also be made and combined with any phosphate modification in the preparation of an ISS.

The heterocyclic bases, or nucleic acid bases, which are incorporated in the ISS can be the naturally-occurring principal purine and pyrimidine bases, (namely uracil or thymine, cytosine, adenine and guanine, as mentioned above), as well as naturally-occurring and synthetic modifications of said principal bases.

Those skilled in the art will recognize that a large number of “synthetic” non-natural nucleosides comprising various heterocyclic bases and various sugar moieties (and sugar analogs) are available in the art, and that as long as other criteria of the present invention are satisfied, the ISS can include one or several heterocyclic bases other than the principal five base components of naturally-occurring nucleic acids. Preferably, however, the heterocyclic base in the ISS includes, but is not limited to, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2,3-d] pyrimidin-5-yl, 2-amino-4-oxopyrrolo [2.3-d] pyrimidin-3-yl groups, where the purines are attached to the sugar moiety of the ISS via the 9-position, the pyrimidines via the 1-position, the pyrrolopyrimidines via the 7-position and the pyrazolopyrimidines via the 1-position.

The ISS may comprise at least one modified base as described, for example, in the commonly owned international application WO 99/62923. As used herein, the term “modified base” is synonymous with “base analog”, for example, “modified cytosine” is synonymous with “cytosine analog.” Similarly, “modified” nucleosides or nucleotides are herein defined as being synonymous with nucleoside or nucleotide “analogs.” Examples of base modifications include, but are not limited to, addition of an electron-withdrawing moiety to C-5 and/or C-6 of a cytosine of the ISS. Preferably, the electron-withdrawing moiety is a halogen. Such modified cytosines can include, but are not limited to, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, uracil, and any other pyrimidine analog or modified pyrimidine.

The preparation of base-modified nucleosides, and the synthesis of modified oligonucleotides using said base-modified nucleosides as precursors, has been described, for example, in U.S. Pat. Nos. 4,910,300, 4,948,882, and 5,093,232. These base-modified nucleosides have been designed so that they can be incorporated by chemical synthesis into either terminal or internal positions of an oligonucleotide. Such base-modified nucleosides, present at either terminal or internal positions of an oligonucleotide, can serve as sites for attachment of a peptide or other antigen. Nucleosides modified in their sugar moiety have also been described (including, but not limited to, e.g., U.S. Pat. Nos. 4,849,513, 5,015,733, 5,118,800, 5,118,802) and can be used similarly.

The ISS used in the methods of the invention may be produced as ISS-microcarrier complexes. ISS-microcarrier complexes comprise an ISS-containing polynucleotide bound to a microcarrier (MC). ISS-MC complexes comprise an ISS bound to the surface of a microcarrier (i.e., the ISS is not encapsulated in the MC), adsorbed within a microcarrier (e.g., adsorbed to PLGA beads), or encapsulated within a MC (e.g., incorporated within liposomes).

ISS-containing oligonucleotides bound to microparticles (SEPHAROSE® beads) have previously been shown to have immunostimulatory activity in vitro (Liang et al., (1996), J. Clin. Invest. 98:1119-1129). However, recent results show that ISS-containing oligonucleotides bound to gold, latex and magnetic particles are not active in stimulating proliferation of 7TD1 cells, which proliferate in response to ISS-containing oligonucleotides (Manzel et al., (1999), Antisense Nucl. Acid Drug Dev. 9:459-464).

Microcarriers are not soluble in pure water, and are less than about 50-60 μm in size, preferably less than about 10 μm in size, more preferably from about 10 nm to about 10 μm, 25 nm to about 5 μm, 50 nm to about 4.5 μm or 1.0 μm to about 2.0 μm in size. Microcarrers may be any shape, such as spherical, ellipsoidal, rod-shaped, and the like, although spherical microcarriers are normally preferred. Preferred microcarriers have sizes of or about 50 nm, 200 nm, 1 μm, 1.2 μm, 1.4 μm, 1.5 μm, 1.6 μm, 1.8 μm, 2.0 μm, 2.5 μm or 4.5 μm. The “size” of a microcarier is generally the “design size” or intended size of the particles stated by the manufacturer. Size may be a directly measured dimension, such as average or maximum diameter, or may be determined by an indirect assay such as a filtration screening assay. Direct measurement of microcarrier size is typically carried out by microscopy, generally light microscopy or scanning electron microscopy (SEM), in comparison with particles of known size or by reference to a micrometer. As minor variations in size arise during the manufacturing process, microcarriers are considered to be of a stated size if measurements show the microcarriers are±about 5-10% of the stated measurement. Size characteristics may also be determined by dynamic light scattering. Alternately, microcarrier size may be determined by filtration screening assays. A microcarrier is less than a stated size if at least 97% of the particles pass through a “screen-type” filter (i.e., a filter in which retained particles are on the surface of the filter, such as polycarbonate or polyethersulfone filters, as opposed to a “depth filter” in which retained particles lodge within the filter) of the stated size. A microcarrier is larger than a stated size if at least about 97% of the microcarrier particles are retained by a screen-type filter of the stated size. Thus, at least about 97% microcarriers of about 10 μm to about 10 nm in size pass through a 10 μm pore screen filter and are retained by a 10 nm screen filter.

As above discussion indicates, reference to a size or size range for a microcarrier implicitly includes approximate variations and approximations of the stated size and/or size range. This is reflected by use of the term “about” when referring to a size and/or size range, and reference to a size or size range without reference to “about” does not mean that the size and/or size range is exact.

Microcarriers may be solid phase (e.g., polystyrene beads) or liquid phase (e.g., liposomes, micelles, or oil droplets in an oil and water emulsion). Liquid phase microcarriers include liposomes, micelles, oil droplets and other lipid or oil-based particles. One preferred liquid phase microcarrier is oil droplets within an oil-in-water emulsion. Preferably, oil-in-water emulsions used as microcarriers comprise biocompatible substituents such as squalene. Liquid phase microcarriers are normally considered nonbiodegradable, but may be biodegradable liquid phase microcarriers may be produced by incorporation of one or more biodegradable polymers in the liquid microcarrier formulation. In one preferred embodiment, the microcarrier is oil droplets in an oil-in-water emulsion prepared by emulsification of squalene, sorbitan trioleate, TWEEN 80® in an aqueous pH buffer.

Solid phase microcarriers for use in ISS-microcarrier complexes may be made from biodegradable materials or nonbiodegradable materials, and may include or exclude agarose or modified agarose microcarriers. Useful solid phase biodegradable microcarriers include, but are not limited to: biodegradable polyesters, such as poly(lactic acid), poly(glycolic acid), and copolymers (including block copolymers) thereof, as well as block copolymers of poly(lactic acid) and poly(ethylene glycol); polyorthoesters such as polymers based on 3,9-diethylidene-2,4,8,10-tetraoxaspiro[5.5]undecane (DETOSU); polyanhydrides such as poly(anhydride) polymers based on sebacic acid, p-(carboxyphenoxy)propane, or p-(carboxyphenoxy)hexane; polyanhydride imides, such as polyanhydride polymers based on sebacic acid-derived monomers incorporating amino acids (i.e., linked to sebacic acid by imide bonds through the amino-terminal nitrogen) such as glycine or alanine; polyanhydride esters; polyphosphazenes, especially poly(phosphazenes) which contain hydrolysis-sensitive ester groups which can catalyze degradation of the polymer backbone through generation of carboxylic acid groups (Schacht et al. (1996) Biotechnol. Bioeng. 1996:102); and polyamides such as poly(lactic acid-co-lysine). A wide variety of nonbiodegradable materials suitable for manufacturing microcarriers are also known, including, but not limited to polystyrene, polyethylene, latex, gold, and ferromagnetic or paramagnetic materials. Solid phase microcarriers may be covalently modified to incorporate one or more moieties for use in linking the ISS, for example by addition of amine groups for covalent linking using amine-reactive crosslinkers.

The ISS-microcarrier complexes of the invention may be covalently or non-covalently linked. Covalently linked ISS-MC complexes may be directly linked or be linked by a crosslinking moiety of one or more atoms (typically the residue of a crosslinking agent). The ISS may be modified to allow or augment binding to the MC (e.g., by incorporation of a free sulfhydryl for covalent crosslinking or addition of a hydrophobic moieties such as lipids, steroids, sterols such as cholesterol, and terpenes, for hydrophobic bonding), although unmodified ISS may be used for formation of non-covalent ISS-MC complex formation by electrostatic interaction or by base pairing (e.g., by base pairing at least one portion of the ISS with a complementary oligonucleotide bound to the microcarrier). ISS-containing polynucleotides may be linked to solid phase microcarriers or other chemical moieties to facilitate ISS-MC complex formation using conventional technology known in the art, such as use of available heterobifunctional crosslinkers (e.g., succinimidyl 4-(N-maleimidomethyl)cyclohexane-1-carboxylate or its sulfo-derivatives for covalently linking an amine-derivatized microcarrier and an ISS modified to contain a free sulfhydryl) or by addition of compounds such as cholesterol (e.g., by the method of Godard et al. (1995) Eur. J. Biochem. 232:404-410) to facilitate binding to hydrophobic microcarriers such as oil droplets in oil-in-water emulsions. Alternatively, modified nucleosides or nucleotides, such as are known in the art, can be incorporated at either terminus, or at internal positions in the ISS. These can contain blocked functional groups which, when deblocked, are reactive with a variety of functional groups which can be present on, or attached to, the microcarrier or a moiety which would facilitate binding to a microcarrier. Certain embodiments of noncovalently linke ISS-MC complexes utilize a binding pair (e.g., an antibody and its cognate antigen or biotin and streptavidin or avidin), where one member of the binding pair is bound to the ISS and the microcarrier is derivatized with the other member of the binding pair (e.g., a biotinylated ISS and a streptavidin-derivatized microcarrier may be combined to form a noncovalently linked ISS-MC complex).

Non-covalent ISS-MC complexes bound by electrostatic binding typically exploit the highly negative charge of the polynucleotide backbone. Accordingly, microcarriers for use in non-covalently bound ISS-MC complexes are generally positively charged at physiological pH (e.g., about pH 6.8-7.4). The microcarrier may intrinsically possess a positive charge, but microcarriers made from compounds not normally possessing a positive charge may be derivatized or otherwise modified to become positively charged. For example, the polymer used to make the microcarrier may be derivatized to add positively charged groups, such as primary amines. Alternately, positively charged compounds may be incorporated in the formulation of the microcarrier during manufacture (e.g., positively charged surfactants may be used during the manufacture of poly(lactic acid)/poly(glycolic acid) copolymers to confer a positive charge on the resulting microcarrier particles.

Solid phase microspheres are prepared using techniques known in the art. For example, they can be prepared by emulsion-solvent extraction/evaporation technique. Generally, in this technique, biodegradable polymers such as polyanhydrates, poly(alkyl-α-cyanoacrylates) and poly(α-hydroxy esters), for example, poly(lactic acid), poly(glycolic acid), poly(D,L-lactic-co-glycolic acid) and poly(caprolactone), are dissolved in a suitable organic solvent, such as methylene chloride, to constitute the dispersed phase (DP) of emulsion. DP is emulsified by high-speed homogenization into excess volume of aqueous continuous phase (CP) that contains a dissolved surfactant, for example, polyvinylalcohol (PVA) or polyvinylpirrolidone (PVP). Surfactant in CP is to ensure the formation of discrete and suitably-sized emulsion droplet. The organic solvent is then extracted into the CP and subsequently evaporated by raising the system temperature. The solid microparticles are then separated by centrifugation or filtration, and dried, for example, by lyophilization or application of vaccum, before storing at 4° C.

Generally, to prepare cationic microspheres, cationic lipids or polymers, for example, 1,2-dioleoyl-1,2,3-trimethylammoniopropane (DOTAP), cetyltrimethylammonium bromide (CTAB) or polylysine, are added either to DP or CP, as per their solubility in these phases.

Physico-chemical characteristics such as mean size, size distribution and surface charge of dried microspheres may be determined. Size characteristics are determined, for example, by dynamic light scattering technique and the surface charge was determined by measuring the zeta potential.

Generally, ISS-containing polynucleotides can be adsorbed onto the cationic microspheres by overnight aqueous incubation of ISS and the particles at 4° C. Microspheres are characterized for size and surface charge before and after ISS association. Selected batches may then evaluated for activity as described herein.

Administration

An ISS-containing polynucleotide may be administered before, during, and/or after exposure to a virus. An ISS polynucleotide may also be administered before, during, and/or after infection by a virus. An ISS-containing polynucleotide may also be administered before or after onset of a symptom of virus infection. Accordingly, administration of ISS-containing polynucleotide may be at various times with respect to exposure to, infection by or onset of symptoms of infection by virus. Further, there may be one or more administrations. If the ISS-containing polynucleotide is administered on multiple occasions, the ISS may be administered on any schedule selected by the clinician, such as daily, every other day, every three days, every four days, every five days, every six days, weekly, biweekly, monthly or at ever longer intervals (which may or may not remain the same during the course of treatment). Where multiple administrations are given, the ISS-containing polynucleotide may be given in 2, 3, 4, 5, 6, 7, 8, 9, 10 or more separate administrations.

When ISS-containing polynucleotide is administered to an individual at risk of exposure to virus (i.e., before infection), ISS-containing polynucleotide is preferably administered less than about 14 days before exposure to virus, preferably less than about 10 days before exposure to virus, more preferably less than about 7 days before exposure to virus, even more preferably less than about 5 days before exposure to virus. In some embodiments, ISS-containing polynucleotide is administered about 3 days before exposure to virus.

In other embodiments, the ISS-containing polynucleotide is administered as soon as possible following a known exposure (e.g., after a needle stick or other percutaneous exposure to a bodily fluid or other material known or thought to be contaminated with virus). In such embodiments, the ISS-containing polynucleotide is preferably administered within 48, 36, 24, or 12 hours after exposure.

In a further embodiment, the ISS-containing polynucleotide is administered after exposure to a virus and before the appearance of any symptoms. This embodiment is particularly relevant with respect to viruses that can take many years between exposure to virus and appearance of symptoms. For example, infection with lentiviruses such as HIV often have an asymptomatic period of up to 20 years before the precipitous drop of CD4 counts in the individual. Another example is infection with papillomavirus which can remain asymptomatic for many years before the presentation of lesions and/or cellular transformations to carcinoma. Preferably, the ISS-containing polynucleotide is administered less than about three days after exposure, more preferably less than about one day, 12 hours, six hours or two hours after exposure, if the time of exposure is known or suspected.

In a further embodiment, the ISS-containing polynucleotide is administered after infection with virus and before the appearance of any symptoms. This embodiment is particularly relevant with respect to viruses that can take many years between infection with virus(es) and appearance of symptoms.

In another embodiment, the ISS-containing polynucleotide is administered upon or after the appearance of one or more symptoms of viral infection. Preferably, ISS-containing polynucleotide is administered within about 28, 21, 14, 7, 5 or 3 days following appearance of a symptom of viral infection. However, some infected individuals exhibiting symptoms will already have undertaken one or more courses of treatment with another therapy (e.g., interferon-based therapy). In such individuals, or in individuals who failed to appreciate the import of their symptoms, the ISS-containing polynucleotide may be administered at any point following infection. Symptoms, described above, will vary depending on the type of virus(es) exposed to the individual. The identification of symptoms is readily accomplished by a skilled clinician.

Additionally, treatments employing an ISS-containing polynucleotide may also be employed in conjunction with other treatments or as ‘second line’ treatments employed after failure of a ‘first line’ treatment.

ISS polynucleotides may be formulated in any form known in the art, such as dry powder, semi-solid or liquid formulations. For parenteral administration ISS polynucleotides preferably administered in a liquid formulation, although solid or semi-solid formulations may also be acceptable, particularly where the ISS polynucleotide is formulated in a slow release depot form. ISS polynucleotides are generally formulated in liquid or dry powder form for topical administration, although semi-solid formulations may occasionally be useful.

ISS polynucleotide formulations may contain additional components such as salts, buffers, bulking agents, osmolytes, antioxidants, detergents, surfactants and other pharmaceutically-acceptable excipients as are known in the art. Generally, liquid ISS polynucleotide formulations made in USP water for injection and are sterile, isotonic and pH buffered to a physiologically-acceptable pH, such as about pH 6.8 to 7.5.

ISS-containing polynucleotides may be formulated in delivery vehicles such as liposomes, oil/water emulsion or slow release depot formulations. Methods of formulating polynucleotides in such forms are well known in the art.

ISS-containing polynucleotide formulations may also include or exclude immunomodulatory agents such as adjuvants and immunostimulatory cytokines, which are well known in the art.

A suitable dosage range or effective amount is one that provides the desired reduction of symptom(s) and/or suppression of viral infection and depends on a number of factors, including the particular virus, ISS sequence of the polynucleotide, molecular weight of the polynucleotide and route of administration. Dosages are generally selected by the physician or other health care professional in accordance with a variety of parameters known in the art, such as severity of symptoms, history of the patient and the like. Generally, for an ISS-containing polynucleotide of about 20 bases, a dosage range may be selected from, for example, an independently selected lower limit such as about 0.1, 0.25, 0.5, 1, 2, 5, 10, 20, 30 40, 50 60, 80, 100, 200, 300, 400 or 500 μg/kg up to an independently selected upper limit, greater than the lower limit, of about 60, 80, 100, 200, 300, 400, 500, 750, 1000, 1500, 2000, 3000, 4000, 5000, 6000, 7000, 8000, 9000 or 10,000 μg/kg. For example, a dose may be about any of the following: 0.1 to 100 μg/kg, 0.1 to 50 μg/kg, 0.1 to 25 μ/kg, 0.1 to 10 μg/kg, 1 to 500 μg/kg, 100 to 400 μg/kg, 200 to 300 μg/kg, 1 to 100 μg/kg, 100 to 200 μg/kg, 300 to 400 μg/kg, 400 to 500 μg/kg, 500 to 1000 μg/kg, 500 to 5000 μg/kg, or 500 to 10,000 μg/kg. Generally, parenteral routes of administration require higher doses of ISS compared to more direct application to infected tissue, as do ISS-containing polynucleotides of increasing length.

Polynucleotides comprising an ISS may be administered by systemic (e.g., parenteral) or local (e.g., topical or intralesional injection) administration.

In one embodiment, the ISS-containing polynucleotide(s) is topically administered. Topical administration may be at the site of infection (e.g., genital region in the case of papillomavirus or herpes simplex virus or respiratory mucosa in the case of respiratory virus), or it may be at a site of a symptom (e.g., papilloma lesion or genital wart).

In another embodiment, the ISS-containing polynucleotide(s) is injected locally into the area of lesion(s). Local injection may be at the site of infection (e.g., genital region in the case of mucosal papillomavirus or herpes simplex virus or into the portal vein in the case of hepatitis virus), site of dysplasia (e.g. epithelium in the genital region), or it may be at a site of a symptom (e.g., intralesionally into a papilloma lesion). Because respiratory viruses infect cells of the respiratory tract, routes which deliver ISS polynucleotides to the respiratory tract, such as inhalation and intranasal delivery (discussed below), are considered local routes of administration in the case of respiratory viruses rather than systemic routes of administration, even though delivery through such routes are normally considered parenteral, systemic routes of administration.

In other embodiments, the ISS-containing polynucleotide is administered systemically such as by parenteral administration. Parenteral routes of administration include but are not limited to transdermal, transmucosal, nasopharyngeal, pulmonary, or direct injection. Parenteral administration by injection may be by any parenteral injection route, including but not limited to intravenous (IV), intraperitoneal (IP), intramuscular (IM), subcutaneous (SC), or intradermal (ID) routes. Transdermal and transmucosal administration may be accomplished by, for example, inclusion of a carrier (e.g., dimethylsulfoxide, DMSO), by application of electrical impulses (e.g., iontophoresis) or a combination thereof. A variety of devices are available for transdermal administration which may be used in accordance with the invention.

Nasopharyngeal and pulmonary routes of administration include, but are not limited to, intranasal, inhalation, transbronchial and transalveolar routes. The ISS-containing polynucleotide may thus be administered by inhalation of aerosols, atomized liquids or powders. Devices suitable for administration by inhalation of ISS-containing compositions include, but are not limited to, nebulizers, atomizers, vaporizers, and metered-dose inhalers. Nebulizers, atomizers, vaporizers and metered-dose inhalers filled with or employing reservoirs containing formulations comprising the ISS-containing polynucleotide(s) are among a variety of devices suitable for use in inhalation delivery of the ISS-containing polynucleotide(s). Other methods of delivering to respiratory mucosa include delivery of liquid formulations, such as by nose drops.

IV, IP, IM, and ID administration may be by bolus or infusion administration. For SC administration, administration may be by bolus, infusion, or by implantable device, such as an implantable minipump (e.g., osmotic or mechanical minipump) or slow release implant. The ISS polynucleotide(s) may also be delivered in a slow release formulation adapted for IV, IP, IM, ID or SC administration. Administration by inhalation is preferably accomplished in discrete doses (e.g., via a metered dose inhaler), although delivery similar to an infusion may be accomplished through use of a nebulizer. Administration via the transdermal and transmucosal routes may be continuous or pulsatile.

Assessment

In some embodiments, administration of an ISS-containing polynucleotide results in prevention, palliation and/or improvement in one or more symptoms of virus infection. The exact form of prevention, palliation or improvement will depend on the particular virus type and the particular symptoms associated with that virus. In some embodiments, administration of an ISS-containing polynucleotide results in a reduction in viral titer (a reduction of which indicates suppression of viral infection). In other embodiments, viral shedding (e.g., virus excretion) is reduced. In some embodiments, the level (e.g., magnitude or amount) of viral shedding is reduced. Viral shedding can occur with or without symptoms at the time of initial or recurrent infection and may be detected, for example, by examination of tissue scrapings from suspected areas of infection for the presence of virus or virus nucleic acid. In other embodiments, viral infection is suppressed, which may be indicated by any one or more of a number of parameters, including, but not limited to, extent of one or more symptoms and viral titer. In other embodiments, recurrence, which is generally indicated by appearance of one or more symptoms associated with infection, is reduced. In other embodiments, the duration of the viral infection is reduced. In other embodiments, one or more physical symptoms (e.g. pain, cachexia, jaundice, breathing difficulties, coughing, etc.) associated with the virus is reduced or improved.

Symptoms of infection may be assessed before or after administration of ISS-containing polynucleotide by the individual or the clinician. As will be apparent to one of skill in the art, the symptoms will vary depending on the particular virus and the site of the symptoms (genital region, oral cavity, respiratory tract, skin, etc.). Symptoms of virus infection can include, but are not limited to, increasing viral titers, fever, pain, declining CD4 count, jaundice, fatigue, lesions, warts, viral shedding, thickening of epithelial layers, pneumonia, cirrhosis and their corresponding symptoms.

Viral titer may be assessed in biological samples using standard methods of the art. Levels of viral nucleic acid may be assessed by isolating nucleic acid from the sample and performing PCR analysis using virus specific primers or blot analysis using a viral polynucleotide sequence as a probe. The PCR analysis can be quantitative using latest PCR technology known in the art. Another method is to perform in situ hybridization with virus-specific probes. Other assays include biological measures such as quantitation of plaque forming units (PFU), infectious center assay (ICA) or virus induced cytopathic effects (CPE), such as formation of syncytia. Extent or amount of viral particles may be measured from any infected area, such as infected tissue or mucosal discharge. When the sample is a liquid, viral titer is calculated in some indication of number or amount of virus or virus particles (e.g., infectious particles, plaque forming units, infectious doses, or median tissue culture infectious doses (TCID 50)) per unit volume. In solid samples, such as a tissue sample, viral titer is calculated in virus particles per unit weight. Reduction is indicated by comparing viral titer to viral titer measured at an earlier time point, and/or comparing to an estimated titer (based, for example, on animal or clinical studies) that represents untreated infection.

Kits of the Invention

The invention provides kits for carrying out the methods of the invention. Accordingly, a variety of kits are provided. The kits may be used for any one or more of the following (and, accordingly, may contain instructions for any one or more of the following uses): preventing one or more symptoms of virus infection in an individual who is at risk of being exposed to a virus; preventing one or more symptoms of virus infection in an individual who has been exposed to a virus; reducing levels of a viral antigen in blood in an individual who has been infected with a virus; reducing viremia in an individual infected with or exposed to a virus; reducing severity of one or more symptoms of virus infection in an individual infected with a virus; reducing recurrence of one or more symptoms of virus infection in an individual infected with a virus; suppressing a virus infection (including reducing viral titer) in an individual infected with or at risk of being infected with a virus; delaying development of a virus infection and/or a symptom of a virus infection in an individual infected or at risk of being infected with a virus; reducing duration of a virus infection in an individual infected or at risk of being infected with a virus. As is understood in the art, any one or more of these uses would be included in instructions directed to treating or preventing a virus infection.

The kits of the invention comprise one or more containers comprising an ISS-containing polynucleotide and a set of instructions, generally written instructions although electronic storage media (e.g., magnetic diskette or optical disk) containing instructions are also acceptable, relating to the use and dosage of the ISS-containing polynucleotide for the intended treatment (e.g., preventing one or more symptoms of virus infection in an individual at risk of being exposed to a virus, preventing one or more symptoms of virus infection in an individual who has been exposed to a virus, reducing severity of one or more symptoms of virus infection in an individual infected with a virus, reducing recurrence of one or more symptoms of virus infection in an individual infected with a virus, suppressing a virus infection in an individual infected with or at risk of being infected with a virus, delaying development of a virus infection and/or a symptom of a virus infection in an individual infected or at risk of being infected with a virus and/or reducing duration of a virus infection in an individual infected or at risk of being infected with a virus). The instructions included with the kit generally include information as to dosage, dosing schedule, and route of administration for the intended treatment. The containers of ISS may be unit doses, bulk packages (e.g., multi-dose packages) or sub-unit doses.

The kits of the invention do not include any packages or containers which include viral antigens from the virus for which the kit is intended to be used to treat. Accordingly, neither the container comprising the ISS nor any other containers in the kit contain viral antigens.

The ISS component of the kit may be packaged in any convenient, appropriate packaging. For example, if the ISS is a freeze-dried formulation, a vial with a resilient stopper is normally used, so that the drug may be easily reconstituted by injecting fluid through the resilient stopper. Vials with non-resilient, removable closures (e.g., sealed glass) or resilient stoppers are most conveniently used for injectable forms of ISS. Also, prefilled syringes may be used when the kit is supplied with a liquid formulation of the ISS-containing polynucleotide. The kit may contain the ISS in an ointment for topical formulation in appropriate packaging. Also contemplated are packages for use in combination with a specific device, such as an inhaler, nasal administration device (e.g., an atomizer) or an infusion device such as a minipump.

As stated above, any ISS-containing polynucleotide described herein may be used, such as, for example, any polynucleotide comprising any of the following ISS: the sequence 5′-C, G, pyrimidine, pyrimidine, C, G-3′, the sequence 5′-purine, purine, C, G, pyrimidine, pyrimidine, C, G-3′, the sequence 5′-purine, purine, C, G, pyrimidine, pyrimidine, C, C-3′; the sequence SEQ ID NO: 1; the sequence 5′-purine, purine, B, G, pyrimidine, pyrimidine-3′ wherein B is 5-bromocytosine or the sequence 5′-purine, purine, B, G, pyrimidine, pyrimidine, C, G-3′ wherein B is 5-bromocytosine.

The following Examples are provided to illustrate, but not limit, the invention.

### EXAMPLES

### Example 1

### Animal Model and Experimental Methods for Respiratory Viruses

Rat Model for RSV Infection and ISS Administration

Cotton rats, 50-100 g and 4-12 weeks old (Sigmoden hispidis) of either sex were used in these studies. All of the animals were descendants of two pair of cotton rats obtained in 1984 from the Small Animal Section of the Veterinary Research Branch, Division of Research Services, National Institutes of Health.

RSV strain A2 was purchased from the ATCC (ATCC VR26). Working stocks of this virus were prepared as described in detail by Wyde et al. (1995) Pediatr. Res. 38:543-550. ISS sequence tested for RSV experiments was 5′-TGACTGTGAACGTTCGAGATGA-3′ (SEQ ID NO:1) (phosphorothioate). Control, non-ISS sequences used were 5′-TGACTGTGAAGGTTAGAGATGA-3′ (SEQ ID NO:9) (phosphorothioate) and 5′-TCACTCTCTTCCTTACTCTTCT-3′ (SEQ ID NO:10) (phosphorothioate), as well as PBS.

Assay for RSV Viral Titer

RSV levels in virus pools and lung lavage fluids (L.F.) were determined using sterile 96-well, flat bottom tissue culture plates (Falcon 3072), serial 3-fold dilutions and 2% FCS-MEM as described in detail previously (Wyde et al., 1995). The wells in these assay plates were observed for virus-induced cytopathic effects (CPE) including formation of synctia. After the dilutions in the last wells of replicate rows exhibiting virus-induced CPE were determined, mean virus titers were calculated using the method of Karber, Rhodes and Van Rhodes and Van Rooyen (1953) Textbook of Virology (2nd ed. Williams and Wilkins pp 66-69). The amount of virus in virus pools was expressed as a median tissue culture infectious doses (TCID₅₀/ml, log₁₀). Titers of virus in L.F. were expressed as TCID₅₀/g lung tissue (log₁₀). The minimum detectable virus concentration in these assays was 1.3 log₁₀ TCID₅₀/ml (virus pools) or 1.6 log₁₀ TCID₅₀/g lung.

### Example 2

### Local Administration of ISS Reduces RSV Viral Titer

These experiments were performed to test the effect of local administration of ISS in terms of antiviral activity against respiratory syncytial virus (RSV) in cotton rats.

On day −3 (i.e., 3 days before infection with virus), 20 cotton rats (CRs) were selected and divided into five groups of four animals. The animals in Group 1 were lightly anesthetized and 50 μL of phosphate buffered saline (PBS) was administered intranasally (IN). The CRs in Group 2 were similarly administered 150 μg of ISS (5′-TGACTGTGAACGTTCGAGATGA-3′) (SEQ ID NO:1), while the animals in Group 3 were similarly administered 150 μg of control non-ISS sequence 5′-TGACTGTGAAGGTTAGAGATGA-3′ (SEQ ID NO:9). Three days later, on Day 0, each of CRs in Group 4 were anesthetized and 150 μg of ISS was administered IN, and the animals in Group 5 were administered, in a like manner, 150 μg of control non-ISS sequence 5′-TGACTGTGAAGGTTAGAGATGA-3′ (SEQ ID NO:9).

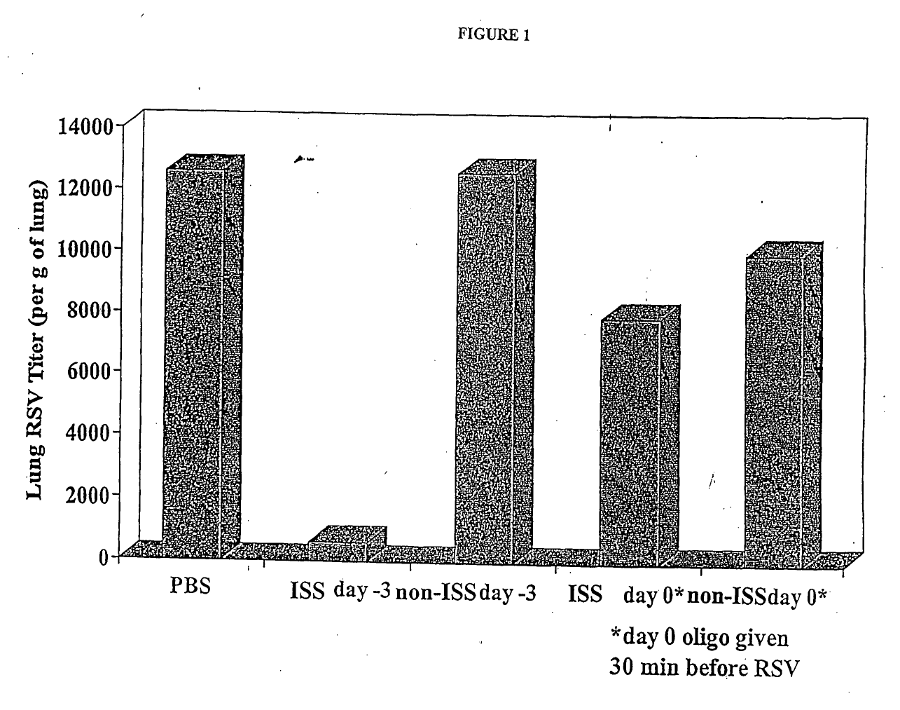

Thirty minutes later, all of the CRs were inoculated IN with 100 median tissue culture infectious doses (TCID₅₀) of RSV A2. Four days later (Day 4), all of the animals were sacrificed and the lungs of each animal were removed, lavaged, and assessed for RSV levels. A summary of the protocol is shown in Table 1. The results are shown in FIG. 1 and Table 2.

TABLE 1
Protocol

| | | Dose | | | | |
|---|---|---|---|---|---|---|
| | | ISS | Day | Day | | |
| | ISS | given | ISS | RSV | Day CRs | |
| Group | admin. | (μg/CR) | given | given | harvested | End-point |
| 1 | PBS | 0 | Day −3 | Day 0 | Day 4 | RSV in lung |
| 2 | ISS | 150 | Day −3 | Day 0 | Day 4 | RSV in lung |
| 3 | non-ISS | 150 | Day −3 | Day 0 | Day 4 | RSV in lung |
| 4 | ISS | 150 | Day 0 | Day 0 | Day 4 | RSV in lung |
| 5 | non-ISS | 150 | Day 0 | Day 0 | Day 4 | RSV in lung |

TABLE 2
RSV Titers

| | | | RSV titer (log₁₀/ | | | | | |
|---|---|---|---|---|---|---|---|---|
| | | | g lung) in CR No. | | | | | |
| Group | Treatment | Day given | 1 | 2 | 3 | 4 | Mean | Std. Dev. |
| 1 | PBS | −3 | 4.5 | 4.5 | 3.5 | 4 | 4.1 | 0.5 |
| 2 | ISS | −3 | 3 | 3 | 2.5 | 2.5 | 2.8 | 0.3 |
| 3 | non-ISS | −3 | 4.5 | 4.5 | 3.5 | 4 | 4.1 | 0.5 |
| 4 | ISS | 0 | 4 | 4 | 4.5 | 3 | 3.9 | 0.6 |
| 5 | non-ISS | 0 | 4.5 | 4 | 4.5 | 3 | 4.0 | 0.7 |

Using the Kruskall-Wallis nonparametric ANOVA p = 0.061, not quite statistically significant.

These results indicate that administration of ISS reduced viral titer in infected tissue compared to PBS or non-ISS administration. The results also indicate that a first administration of ISS on the day of infection was not effective, while administration before infection (in this experiment, 3 days) was effective at reducing viral titers.

### Example 3

### Non-local Administration of ISS and RSV Viral Titer

These experiments were performed to test the effect of non-local administration of ISS in terms of antiviral activity against RSV in cotton rats.

Twenty cotton rats were divided into 5 groups of 4 animals. Administered to these animals, either intraperitoneally (IP) or subcutaneously (SC), was PBS, immunostimulatory sequence (ISS) 5′-TGACTGTGAACGTTCGAGATGA-3′ (SEQ ID NO:1) or non-ISS sequence 5′-TCACTCTCTTCCTTACTCTTCT-3′ (SEQ ID NO:10), each sequence at 150 μg/injection. On Day 0 each of these animals was ated IN with 100 TCID₅₀ of RSV A2. Four days later each cotton rat was sacrificed. The lungs of each animal were removed, lavaged and assessed for RSV. The protocol is summarized in Table 3. The results from IP administration are shown in Table 4. The results from SC administration are shown in Table 5.

TABLE 3
Protocol

| | | Dose ISS | Day | Day | | |
|---|---|---|---|---|---|---|
| | ISS | given | ISS | RSV | Day CRs | |
| Group | admin. | (μg/CR) | given | given | Sacrificed | End-point |
| 1 | PBS | 0 | −3, −1 | 0 | Day 4 | RSV in lung |
| 2 | ISS | 150 | −1 | 0 | Day 4 | RSV in lung |
| 3 | ISS | 150 | −3 | 0 | Day 4 | RSV in lung |
| 4 | non-ISS | 150 | −1 | 0 | Day 4 | RSV in lung |
| 5 | non-ISS | 150 | −3 | 0 | Day 4 | RSV in lung |

TABLE 4
RSV Titers

| | | | RSV titer (log₁₀/ | | | | | |
|---|---|---|---|---|---|---|---|---|
| | Treatment | Day (s) | g lung) in cotton rat no. | | | | | Std. |
| Group | (IP) | given | 1 | 2 | 3 | 4 | Mean | Dev. |
| 1 | PBS | −1, −3 | 4.3 | 3.8 | 3.8 | 3.3 | 3.8 | 0.3 |
| 2 | ISS | −1 | 3.8 | 3.3 | 3.3 | 3.8 | 3.6 | 0.3 |
| 3 | ISS | −3 | 3.3 | 3.8 | 3.8 | 3.8 | 3.7 | 0.3 |
| 4 | Non-ISS | −1 | 1.8 | 3.3 | 3.8 | 3.3 | 3.1 | 0.9 |
| 5 | Non-ISS | −3 | 3.3 | 4.3 | 3.3 | 3.3 | 3.6 | 0.5 |

TABLE 5
RSV titers

| | | | RSV titer (log₁₀/ | | | | | |
|---|---|---|---|---|---|---|---|---|
| | Treatment | Days | g lung) in CR no. | | | | | Std. |
| Group | (SC) | given | 1 | 2 | 3 | 4 | Mean | Dev. |
| 1 | PBS | −1, −3 | 4 | 4 | 3.5 | 4 | 3.9 | 0.3 |
| 2 | ISS | −1 | 4 | 4.5 | 3.5 | 4 | 4.0 | 0.4 |
| 3 | ISS | −3 | 4 | 4.5 | 4 | 4 | 4.1 | 0.3 |
| 4 | Non-ISS | −1 | 4.5 | 4.5 | 3.5 | 4 | 4.1 | 0.5 |
| 5 | Non-ISS | −3 | 3.5 | 4 | 4 | 3.5 | 3.8 | 0.3 |

In each experiment, IP and SC administration of 150 μg of ISS-containing polynucleotide failed to cause a statistically significant reduction in viral titers compared to PBS administration.

### Example 4

### Local Administration of ISS and Influenza Viral Titer

These experiments were performed to test the effect of local administration of ISS in terms of antiviral activity against influenza virus in mice.

Thirty-five mice were divided into 5 groups of 7 animals each. On Day −3 (relative to virus inoculation), PBS (50 μl) was administered intranasally (IN) to the animals in Group 1, while ISS 5′-TGACTGTGAACGTTCGAGATGA-3′ (SEQ ID NO:1) was administered IN (50 μg in 50 μl/mouse) to the animals in Group 2 and non-ISS control sequence 5′-TGACTGTGAAGGTTAGAGATGA-3′ (SEQ ID NO:9) was administered IN (50 μg in 50 μl/mouse) to the animals in Group 3. Three days later (Day 0), ISS (50 μg/mouse) or non-ISS control of sequence (50 μg/mouse) were administered IN to the animals in Groups 4 and 5, respectively. On day 0, 50 μl of PBS was administered IN to the animals in Group 1. Shortly after these administrations on day 0, all of the mice were inoculated IN with approximately 100 median tissue culture infectious doses (TCID₅₀) of influenza A/Mississippi (H3N2) virus. Four days later, all of the mice were sacrificed and the lungs of each were tested for influenza virus titer. The protocol is summarized in Table 6. The results are summarized in Table 7. The results show that IN administration of this dose of ISS before viral infection fails to cause a satisfactory significant reduction in virus titer compared to PBS administration.

TABLE 6
Protocol

| | | Day | Virus | Day | Test |
|---|---|---|---|---|---|
| Group | Treatment | given | inoc. | Sacrifice | parameter |
| 1 | PBS | −3, 0 | Day 0 | Day 4 | Pulmonary |
| 2 | ISS | −3 | Day 0 | Day 4 | virus |
| 3 | non-ISS | −3 | Day 0 | Day 4 | titer |
| 4 | ISS | 0 | Day 0 | Day 4 | |
| 5 | non-ISS | 0 | Day 0 | Day 4 | |

TABLE 7
Influenza Virus Titers

| | | Day | Pulmonary virus titer | | | | | | | | |
|---|---|---|---|---|---|---|---|---|---|---|---|
| | Treat- | ISS | (log₁₀/lung) in mouse no. | | | | | | | | Std. |
| Group | ment | given | 1 | 2 | 3 | 4 | 5 | 6 | 7 | Mean | Dev. |
| 1 | PBS | −3, 0 | 3.5 | 4 | 4.5 | 6 | 4.5 | 4.5 | 4 | 4.4 | 0.8 |
| 2 | ISS | −3 | 5.5 | 4 | 6 | 5.5 | 5 | 4 | 3 | 4.7 | 1.1 |
| 3 | non-ISS | −3 | 3.5 | 3.5 | 4 | 3 | 5 | 5 | 4 | 4.0 | 0.8 |
| 4 | ISS | 0 | 4 | 5.5 | 5 | 4.5 | 4.5 | 4.5 | 4.5 | 4.6 | 0.5 |
| 5 | non-ISS | 0 | 5.5 | 4 | 4.5 | 4.5 | 6 | 5.5 | 4.5 | 4.9 | 0.7 |

### Example 5

### Non-local Administration of ISS and Influenza Viral Titer

These experiments were performed to test the effect of non-local administration of ISS in terms of antiviral activity against influenza virus in mice.

Twenty-five mice were divided into 5 groups of 5 animals each. On Day −3 (relative to virus inoculation), PBS was administered intraperitoneally (IP) to the animals in Group 1, while ISS 5′-TGACTGTGAACGTTCGAGATGA-3′ (SEQ ID NO:1) was administered IP (50 μg/mouse) to the animals in Group 3 and non-ISS control sequence 5′-TCACTCTCTTCCTTACTCTTCT-3′ (SEQ ID NO:10) was administered IP (50 μg/mouse) to the animals in Group 5. On Day −1, ISS (50 μg/mouse) or non-ISS control of sequence (50 μg/mouse) were administered IP to the animals in Groups 2 and 4, respectively. The next day (Day 0), all of the mice were inoculated intranasally (IN) with approximately 100 median TCID₅₀ of influenza A/Mississippi (H3N2) virus. Four days later, all of the mice were sacrificed and the lungs of each were tested for influenza virus titer. The protocol is summarized in Table 8. The results are summarized in Table 9. The results show that IP administration of this dose of ISS before viral infection fails to cause a satisfactory significant reduction in virus titer compared to PBS administration.

TABLE 8
Protocol

| | | Day | Virus | Day | Test |
|---|---|---|---|---|---|
| Group | Treatment | given | inoc. | Sacrifice | parameter |
| 1 | PBS | −3, −1 | Day 0 | Day 4 | Pulmonary |
| 2 | ISS | −1 | Day 0 | Day 4 | virus |
| 3 | ISS | −3 | Day 0 | Day 4 | titer |
| 4 | non-ISS | −1 | Day 0 | Day 4 | |
| 5 | non-ISS | −3 | Day 0 | Day 4 | |

TABLE 9
Influenza Virus Titers

| | | | Pulmonary virus titer | | | | | | |
|---|---|---|---|---|---|---|---|---|---|
| | | Day ISS | (log₁₀/lung) in mouse no. | | | | | | Std. |
| Group | Treatment | given | 1 | 2 | 3 | 4 | 5 | Mean | Dev. |
| 1 | None | −3, −1 | 5.8 | 7.3 | 5.8 | 6.3 | 6.3 | 6.3 | 0.6 |
| 2 | ISS | −1 | 6.3 | 6.8 | 7.3 | 6.8 | 6.8 | 6.8 | 0.4 |
| 3 | ISS | −3 | 7.3 | 5.8 | 7.3 | 6.8 | 7.3 | 6.9 | 0.7 |
| 4 | non-ISS | −1 | 6.8 | 6.3 | 5.8 | 5.8 | 5.8 | 6.1 | 0.4 |
| 5 | non-ISS | −3 | 5.8 | 5.8 | 6.3 | 7.3 | 7.3 | 6.5 | 0.8 |

### Example 6

### Administration of an ISS in an Animal Model of Chronic HBV Infection

ISS activity was tested in an animal model of chronic hepatitis. An ISS-containing phosphorothioate oligonucleotide (5′-TGACTGTGAACGTTCGAGATGA-3′) (SEQ ID NO:1), was delivered to STC strain transgenic mice, followed by measurement of HBV DNA and HBsAg production.

STC line mice were developed at Stanford University by Patricia Marion. The majority of these mice secrete HBV of the Ayw genotype (Galibert et al. (1979) Nature 281:646) to titers of 10⁶⁻⁸ viral genome equivalents per ml of serum. STC mice were derived from the FVB strain, and were constructed by microinjection of HBV genomic DNA. STC mice have been shown to be responsive to drugs which inhibit HBV replication, and so are considered a good model of chronic HBV.

Approximately one month old mice were bled and tested for serum levels of HBsAg, which is predictive of viral DNA titer. A pool of 40 STC mice with approximately equal levels of HBsAg were selected and randomly assigned to four treatment groups of 10 animals each. The groups were treated as follows:
1. 100 μg of ISS injected subcutaneously, once per week for 3 weeks (days 0, 7, 14)
2. 100 μg of ISS injected subcutaneously, one injection at day 14
3. 100 ng of murine 1L-12 injected intraperitoneally on days 12, 13, and 14.
4. PBS injected subcutaneously (days 0, 7, 14)

Blood samples were taken at day 0, 7, 14, 15 (22 hr after last IL-12 injection), 18, 28 and 35. Serum prepared from the blood samples was tested for HBV DNA by quantitative PCR (testing performed under contract by Hepadnavirus Testing, Inc.), and HBsAg using a commercially available EIA kit for HBsAg from Abbott Laboratories. Animals were sacrificed at day 35 and livers were collected for histologic analysis.

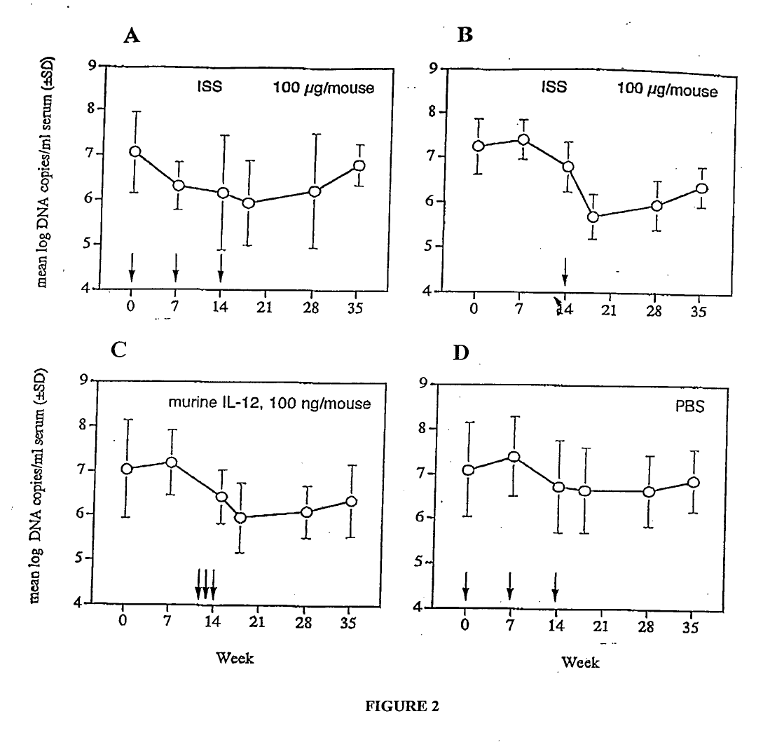

The results of the quantitative PCR assays for serum HBV DNA levels in HBV-producing mice treated with ISS, murine IL-12 or PBS, are summarized in FIG. 2. The results are plotted as means of the HBV DNA levels of each of the 4 groups in each of the serial samples. Samples were blinded to the person conducting the assays. Both ISS and murine IL-12 were effective in reducing viral titer in STC mice. The most dramatic titer drop was seen in Group 2 (single subcutaneous injection of ISS at day 14), where the mean viral DNA titer was reduced by 90 fold three days after injection.

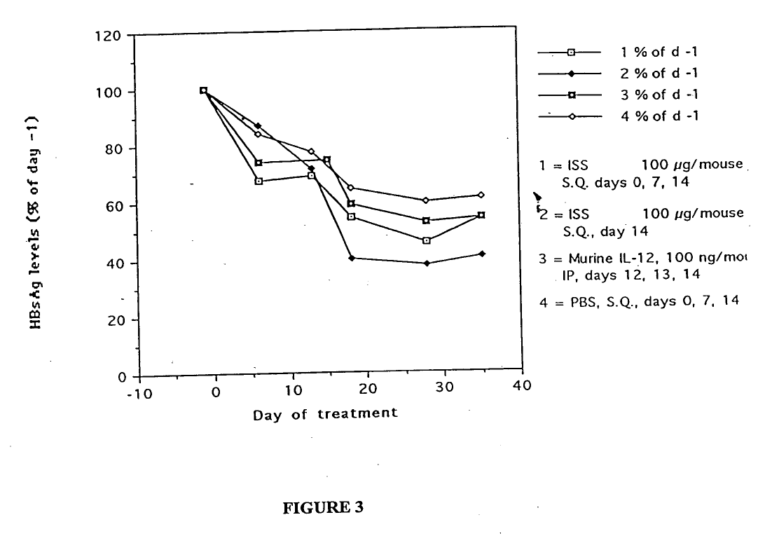

The results of the assays for serum HBsAg levels in HBV-producing mice treated with ISS, murine IL-12 or PBS are summarized in FIG. 3. The results are plotted as averages of the antigen levels of each of the 4 groups in each of the serial sample. The data showed a trend towards decreased average HBsAg values of animals treated with ISS compared to control animals treated with PBS.

It should be noted that, as with all lineages of HBV-producing mice, some animals sharply dropped titer during the observation period, even before treatments, or with treatment with the control. Despite the randomizing at −7 days, more of these mice were found in groups 3 and 4 (IL-12 and control, respectively), possibly obscuring a more dramatic effect by the ISS.

### Example 7

### Delay of HSV Disease Development in Mice by Administration of ISS

Outbred Swiss Webster mice, vaginally infected with HSV-2 strain 186, were used as a model of HSV infection. In these animals, the first indication of viral infection is hair loss and erythema (HLE) near the vagina occurring, on average, 5 days after inoculation. The next stage of infection is indicated by chronic wetness (CW) due to loss of bladder control, on average, 6 days after inoculation. A portion (about 50% of infected mice) of the animals develop hind limb paralysis (HLP) at approximately the same time point. Death, which is often preceded by evidence of CNS disease, occurs an average of 7-9 days after viral inoculation.

Mice were prepared for infection by an initial two-dose treatment with depopriven to synchronize cycles and to thin the vaginal epithelium. Vaginal mucous was removed by swabbing with calcium alginate, then a lethal challenge dose (determined by titration) of HSV-2 strain 186 was delivered by positive-displacement pipettor. Inoculated mice were randomly grouped into one of 4 treatment groups (n=15/group). Animals in group 1 received no treatment and served as a control for the study. Animals in the second and third groups were treated topically with 100 μg of an ISS-containing phosphorothioate oligonucleotide (5′-TGACTGTGAACGTTCGAGATGA-3′) (SEQ ID NO:1) suspended in phosphate-buffered saline (PBS). The groups were treated 2 or 6 hours after inoculation. As a vehicle control, group four was treated with PBS alone.

Treatment with ISS resulted in decreased incidence (i.e., individuals showing symptoms of HSV2 infection), improved survival and delays in both appearance of symptoms and time to death in symptomatic individuals. For those individuals which died during the experiment, average time to death was increased by an average of over two days in animals treated with ISS two hours after infection. Log rank analysis of the data indicated a statistical difference for both ISS treatment times compared to either the no treatment or PBS vehicle-treated groups (p=0.0014 and 0.0146, respectively). The data from this experiment are summarized in Table 10 (PI, post-inoculation).

TABLE 10

| | | | Time | Time |
|---|---|---|---|---|
| Group | Incidence | Survival | to Symptoms | to Death |
| No Treatment | 15/15 (100%) | 0/15 (0%) | 4.73 d | 8.1 d |
| ISS 2 h PI | 9/15 (60%) | 6/15 (40%) | 6.6 d | 12 d |
| ISS 6 h PI | 2/15 (80%) | 4/15 (27%) | 5.75 d | 10.6 d |
| PBS 6 h PI | 15/15 (100%) | 0/15 (0%) | 4.9 d | 9.5 d |

In another experiment, inoculated mice were randomly grouped into 8 treatment groups (n=16/group). Animals in the groups received treatments as outlined in Table 11 below. The groups were treated 2 hours after virus inoculation.

TABLE 11

| | |
|---|---|
| Group | Treatment |

| | | |
|---|---|---|
| 1 | ISS; 5′- | (SEQ ID NO: 1) |
| | TGACTGTGAACGTTCGAGATGA-3′ | |
| 2 | ISS; 5′- | (SEQ ID NO: 11) |
| | TCGTCGAACGTTCGTTAACGTTCG-3′ | |
| 3 + 4 | non-ISS; 5′- | (SEQ ID NO: 9) |
| | TGACTGTGAAGGTTAGAGATGA-3′ | |
| 5 | non-ISS; 5′- | (SEQ ID NO: 12) |
| | TGACTGTGAACCTTAGAGATGA-3′ | |
| 6 | PBS | |
| 7 | No Treatment | |
| 8 | Acyclovir | |

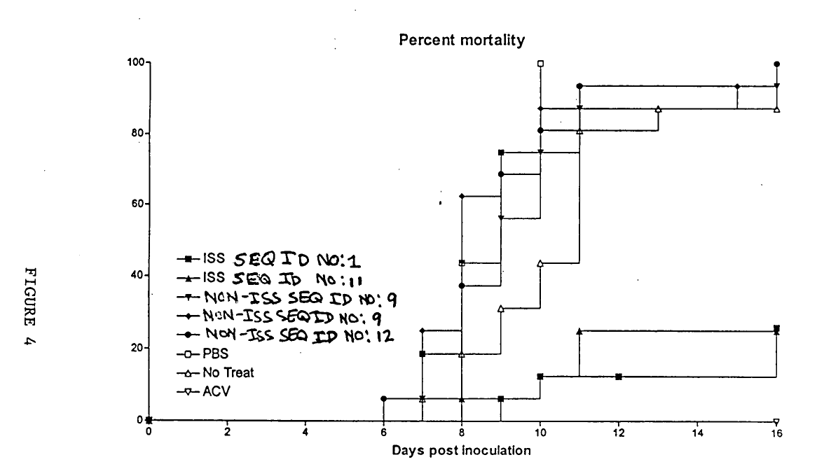

In sum, treatment with ISS resulted in decreased incidence (i.e., individuals showing symptoms of HSV2 infection), improved survival and delays in both appearance of symptoms and time to death in symptomatic individuals. For example, survival results of this experiment are depicted in FIG. 4. The survival curves for the animals treated with the two ISS oligonucleotides are indistinguishable from each other and are both significantly different from those of the other treatment groups.

### Example 8

### Reduction of HSV Lesions in Guinea Pigs by Administration of ISS

Recurrent HSV-2 disease and aspects of the primary disease, including vesicular ulcerative lesion formation and asymptomatic shedding, are effectively modeled by inoculation of the guinea pig vagina with HSV-2 (Milligan et al. (1995) Virol. 206:234-241). In the guinea pig model, animals are infected by instillation of HSV-2 after calcium-alginate swabbing as described in Example 7. Three to five days after inoculation, cutaneous lesions develop and in some cases urinary retention is observed. The animals are scored daily for lesion severity using a 4 point scale (Bourne et al. (1996) J. Infect. Dis. 173:800-807). Primary disease resolves by 14 days after inoculation. From day 15 through 70 after inoculation, the animals are scored daily for the development of recurrent lesions. The frequency of recurrence is a significant outcome measure as it indicates any impact on latency and reactivation that a therapy may have. This model has proved to be a very effective system for testing of antivirals and vaccines (Bourne et al. (1996) Vaccine 14:1230-1234; Stanberry (1989) Antiviral Res. 11:203-214; Stanberry et al. (1990) Antiviral Res. 13:277-286).

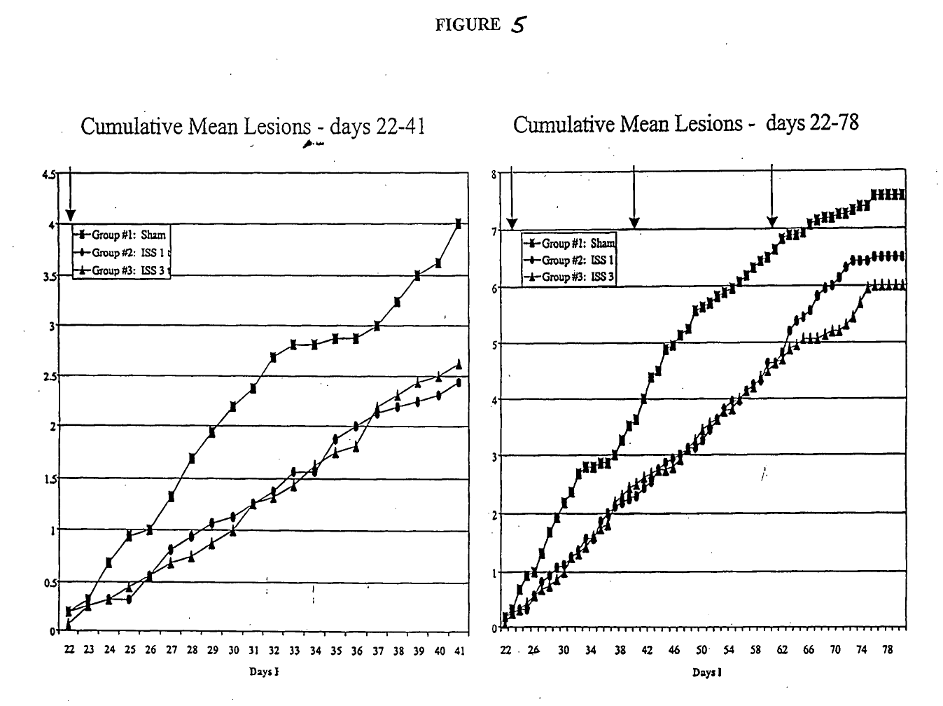

Swiss Hartley guinea pigs (Charles River Laboratories) were intravaginally inoculated with HSV-2 strain MS by simply delivering virus to the vagina, then followed through the primary infection (d14 PI). Animals that did not display herpetic lesions were eliminated from further study. The remaining animals were randomly assigned to one of three study groups (n=16/group). To assess the impact of the ISS therapy upon recurrent lesion development, two of the three study groups were treated with 200 μg of the ISS-containing polynucleotide of Example 7 (5′-TGACTGTGAACGTTCGAGATGA-3′) (SEQ ID NO:1) suspended in PBS 21 days post inoculation. The third group received an injection of PBS alone. One of the two ISS treated groups received two additional ISS injections on days 42 and 63 post-inoculation (PI) (Group #3). Daily scoring of recurrent lesions was completed on each animal to determine the impact of ISS on recurrence frequency. These scores were averaged daily for each groups and the cumulative totals are depicted in FIG. 5. The graph on the left shows the period of time immediately following the first ISS injection (days 22-41), while the graph on the right shows the data over the entire observation period (day 22 through day 78).

Statistical analysis (ANOVA) of the results showed a significant reduction in the frequency of recurrences following ISS therapy (p=0.012). No difference was observed among the groups prior to ISS treatment. Although the results between multiple and single treatments were not statistically significant (p>0.05), data trends suggested that multiple treatments may further reduce recurrences.

In another experiment, guinea pigs were intravaginally inoculated with 5×10⁵ pfu HSV-2, strain MS as described above. Groups of animals were treated with one of the following regimens:

| | |
|---|---|
| Group | Treatment |

| | | |
|---|---|---|
| 1 | ISS; | (SEQ ID NO: 1) |
| | 5′-TGACTGTGAACGTTCGAGATGA-3′; | |
| | 1 mg in PBS; once at 21 days | |
| | post inoculation | |
| 2 | non-ISS; | (SEQ ID NO: 9) |
| | 5′-TGACTGTGAAGGTTAGAGATGA-3′; | |
| | 1 mg in PBS; once at 21 days | |
| | post inoculation | |
| 3 | No Treatment | |
| 4 | Acyclovir; 3 times/day for 7 | |
| | days starting at 6 hours post | |
| | inoculation | |

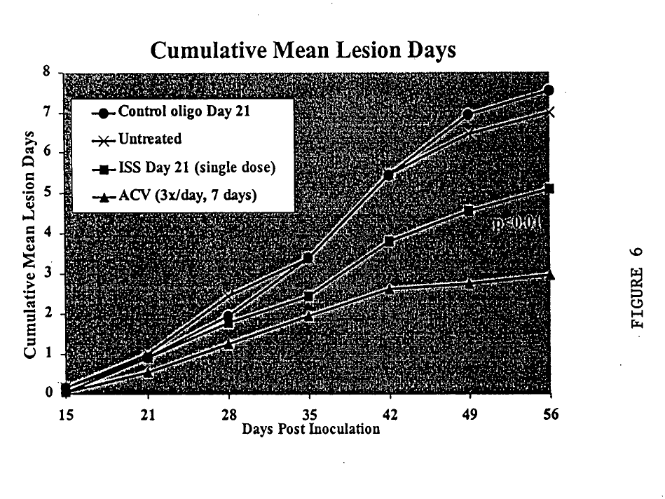

Recurrent disease was monitored from day 15-56 post inoculation. Vaginal swabs of animals were done on days 21-43 and PCR analysis performed to determine the level of viral shedding. To evaluate the effect of ISS therapy on recurrent disease, cumulative number of recurrent lesions were monitored over time and the mean calculated for the group. Results from this experiment are depicted in FIG. 6. A single topical treatment with ISS at day 21 significantly decreased the cumulative mean recurrent lesion days compared to animals treated with non-ISS control oligonucleotide or untreated animals. The acyclovir group also showed a significant reduction in cumulative recurrent mean lesion days, however this group received a total of 21 treatments spread over 7 days to achieve this effect.

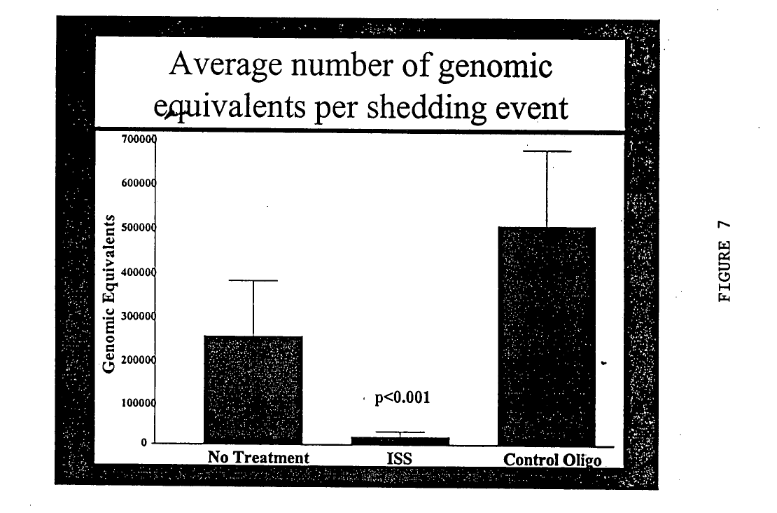

The frequency of viral shedding was 20% of days for all groups. Thus, the frequency of viral shedding was unaffected by ISS treatment. However, as shown in FIG. 7, the magnitude of viral shedding was significantly reduced in the group receiving a single topical treatment with ISS as compared to the control groups. The p value (p<0.001) was calculated by ANOVA analysis using Dunn's Multiple Comparison test and is valid for both the untreated group and the non-ISS control oligonucleotide group. Magnitude of virus shedding is correlated with viral transmission. Since ISS treatment resulted in a reduction in the magnitude of viral shedding, ISS treatment may be effective in a reduction in viral transmission.

### Example 9

### ISS Demonstrates No Direct Activity on Viral Replication

As demonstrated in the following experiment, ISS appears to have no direct activity on viral replication.

Vero cells, a cell line derived from African Green monkey kidney, were pre-treated with varying concentrations of ISS or non-ISS oligonucleotides for varying times prior to the addition of HSV-1 or HSV-2. Oligonucleotides were used at 1 μg/ml or 10 μg/ml and the cells were incubated with the oligonucleotides for 30 seconds, 10 minutes or 24 hours. Viral titers were calculated as a percent of control titer generated by cells not treated with the oligonucleotides. The experimental conditions and results are summarized in Table 12 (NA=not available). The data are expressed as percent of control titer.

TABLE 12

| | 1 μg/ml | | | 10 μg/ml | | |
|---|---|---|---|---|---|---|
| Oligonucleotide | 30 sec | 10 min | 24 hr | 30 sec | 10 min | 24 hr |

Cells infected with HSV-1

| | | | | | | |
|---|---|---|---|---|---|---|
| SEQ ID NO: 1 | 98 | 96 | 89 | 100 | 102 | 82 |
| SEQ ID NO: 11 | 129 | 95 | 87 | 122 | 96 | 78 |
| SEQ ID NO: 12 | 132 | 98 | 97 | 141 | 100 | 94 |
| SEQ ID NO: 9 | 100 | 99 | 101 | 96 | 100 | 97 |

Cells infected with HSV-2

| | | | | | | |
|---|---|---|---|---|---|---|
| SEQ ID NO: 1 | 101 | 98 | 99 | 101 | 101 | 99 |
| SEQ ID NO: 11 | 119 | NA | NA | 136 | NA | NA |
| SEQ ID NO: 12 | 111 | NA | NA | 129 | 100 | 98 |
| SEQ ID NO: 9 | 98 | 96 | 103 | 103 | 97 | 99 |

HSV-1 or HSV-2 virus was pre-treated with varying concentrations of ISS or non-ISS oligonucleotides for 10 minutes prior to adding the mixture to plated Vero cells. Oligonucleotides were used at 1 μg/ml or 10 μg/ml. Viral titers were calculated as a percent of control titer generated by cells not treated with the oligonucleotides. The experimental conditions and results are summarized in Table 13. The data are expressed as percent of control titer.

TABLE 13

| | HSV-1 | | | HSV-2 | | |
|---|---|---|---|---|---|---|
| | 1 | 10 | | 1 | 10 | |
| Oligonucleotide | μg/ml | μg/ml | control | μg/ml | μg/ml | control |
| SEQ ID NO: 1 | 101 | 109 | 100 | 96 | 102 | 100 |
| SEQ ID NO: 11 | 100 | 100 | 99 | 101 | 97 | 99 |
| SEQ ID NO: 12 | 98 | 101 | 100 | 100 | 97 | 103 |
| SEQ ID NO: 9 | 102 | 103 | 102 | 98 | 106 | 101 |

As shown in Tables 12 and 13, incubating the cells with ISS prior to HSV infection in vitro and incubating HSV virus with ISS prior to infecting cells in vitro has no effect on the viral titers from the infected cells as compared to controls.

### Example 10

### Treatment of Canine Oral Papilloma with ISS

A model of canine oral papilloma was used to test the efficacy of ISS on papilloma. Beagle puppies were inoculated in the bucal mucosa with canine papillomavirus and developing papilloma lesions were monitored daily. Four groups of seven dogs each were treated with differing amount of ISS oligonucleotide (5′-TGACTGTGAACGTTCGAGATGA-3′ (SEQ ID NO:1), phosphorothioate backbone). One group received 50 μg ISS twice a week, another group received 500 μg ISS twice a week, the third group received 500 μg ISS one time only at the first signs of papilloma lesion development (injected within the papilloma lesion) and the fourth group (control group) received PBS twice a week. All dogs were monitored daily for the development of lesions and the time to regression.

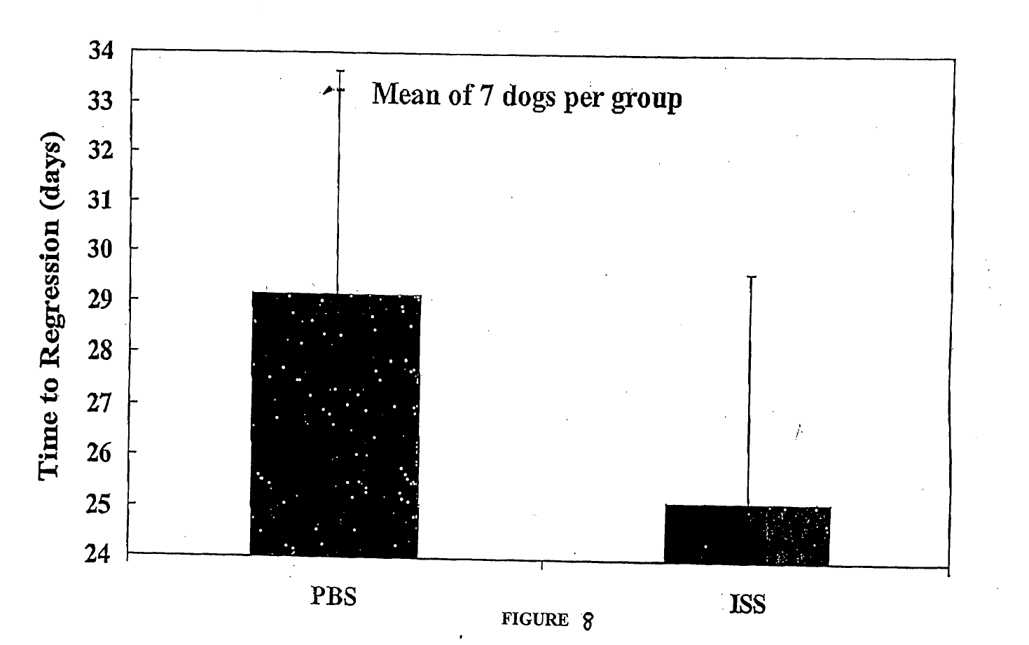

The results are shown in FIG. 8. Dogs that received a one time treatment of 500 μg ISS at the first signs of papilloma lesion showed a higher average rate of lesion regression than untreated dogs, although the ranges for both groups overlapped. Untreated dogs took an average of 29.1 days for rapid regression while dogs treated with 500 μg ISS at the first signs of papilloma took an average of 25.1 days for rapid regression.

The other treatment groups did not show a marked difference in regression time. This model offers a short window of time in which regression of warts can be observed. In dogs, warts caused by canine papillomaviruses can spontaneously regress. Injection of ISS in the papillomas when papillomas first appear appears to enhance the time of lesion regression as compared to the time of spontaneous lesion regression.

### Example 11

### Treatment of Cutaneous Papillomatosis in a Rabbit Model by ISS

Rabbits were initially the first animals in which papillomavirus infection was described in 1933 by Shope. Shope recognized the cottontail rabbit papillomavirus (CRPV) as the etiological agent for cutaneous papillomatosis in the cottontail rabbit (Howley, P., Chapter 65, Fields Virology, Vol. 2, Third Edition, Lippincott-Raven publishers).

In this model of papilloma, New Zealand White rabbits of both genders were quarantined for 14 days, those animals remaining healthy were cleared for use in the experiment. 15 rabbits were each inoculated with a high dose of CRPV at two different sites (one on each side fo the animal) and a low dose of CRPV at two different sites (one on each side fo the animal) for a total of four inoculation sites in each rabbit. The animals were then separated into three groups of five animals each, groups A, B, and C.

Group A received 50 μg intradermal injections of ISS oligonucleotide (5′-TGACTGTGAACGTTCGAGATGA-3′ (SEQ ID NO:1), phosphorothioate backbone) into the site of CRPV inoculation (site of the papilloma lesion at later time points) at Day 1 (one day following inoculation with CRPV) and Day 21 on the left side and at Day 14 and Day 35 on the right side. Groups B and C received intradermal injections of 500 μpg of the ISS and phosphate-buffered saline (vehicle), respectively, into the site of CRPV inoculation (site of the papilloma lesion at later time points) on the same schedule.

Papilloma development was quantitated by finding the geometric mean diameter (GMD) of each papilloma lesion. GMD was calculated from measurements of the length, width and height of the papilloma lesions. Measurements were made weekly.

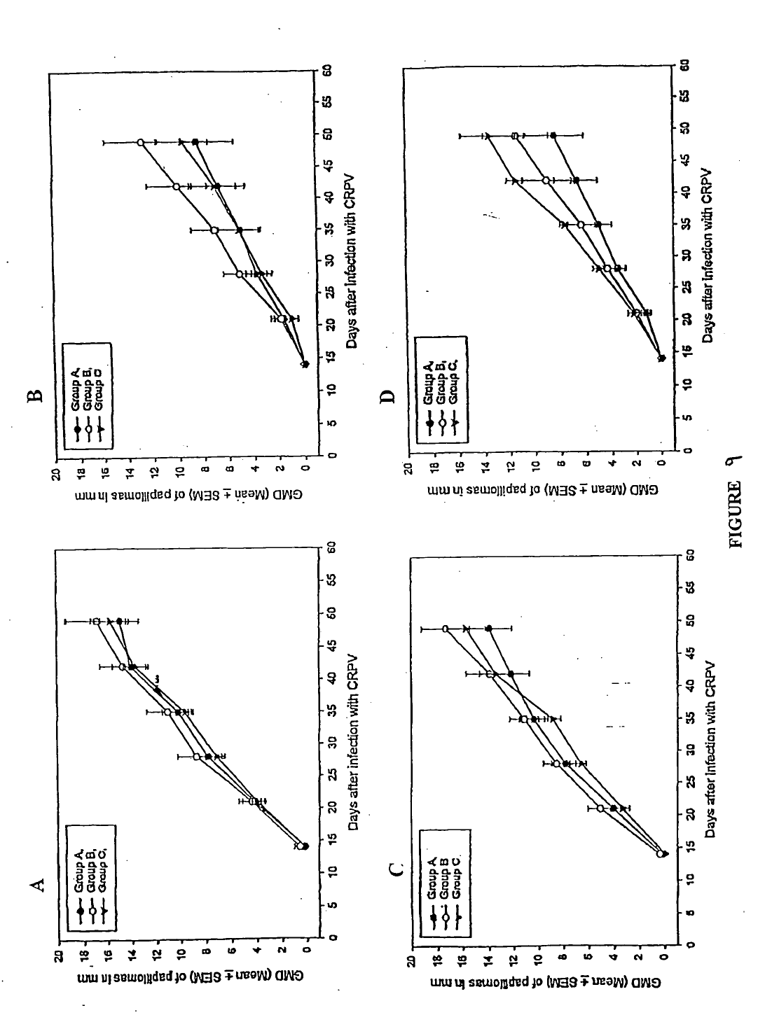

Results are summarized in FIG. 9. Panel A shows GMD for the left side, high CRPV dose lesions (treatment on Day 1 and 14). Panel B shows GMD for the left side, low CRPV dose lesions (treatment on Day 14 and 35). Panel C shows average GMD for the right side, high CRPV dose lesions (treatment on Day 1 and 14). Panel D shows average GMD for the right side, low CRPV dose lesions (treatment on Day 14 and 35).

In another experiment, a mutant of CRPV which induces small papillomas, CRPV-E8m, was used to induce papillomas on five rabbit treatment groups (five rabbits per group). In each animal, papillomas on the left side of the animal received treatments and papillomas on the right side were untreated. Four of the treatment groups received doses between 100 μg and 2000 μg of ISS as intradermal injections per papilloma at several treatment regimes and the fifth group received injections of PBS as control, as outlined below.

| Group | Left Side Treatment |
|---|---|
| A | ISS; 100 μg/injection; 3 times/week from days 47-86 |
| B | ISS; 100 μg/injection; 1 time/week from days 47-86 |
| C | ISS; 500 μg/injection; 1 time/week from days 47-86 |
| D | ISS; 2000 μg/injection; weeks 7 and 10 |
| E | PBS; 100 μl/injection; 3 times/week from days 47-86 |

Four papillomas, initiated with CRPV-E8m plasmid DNA, were established on each rabbit. Skin at the site of papilloma initiation was made hyperplastic using a mixture of turpentine and acetone prior to viral DNA administration. The size of papillomas was measured (three dimensions, in mm) and the GMD calculated for each papilloma.

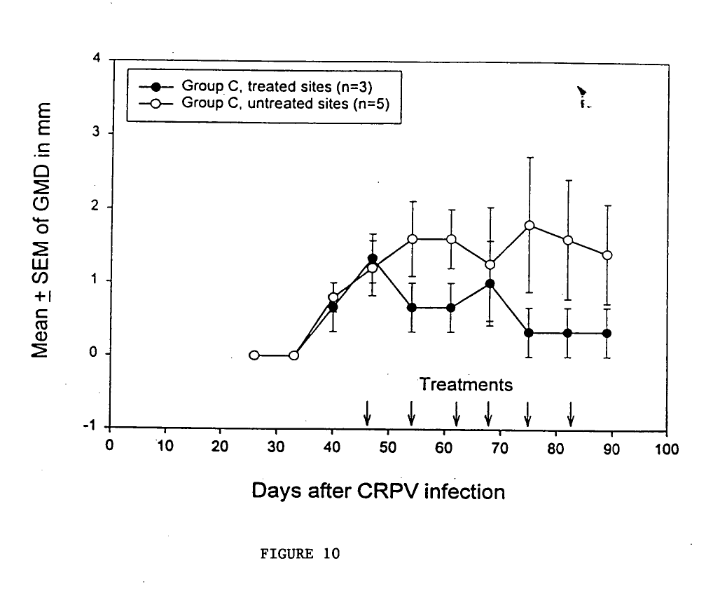

In this experiment, a number of viral DNA challenged sites failed to generate any papillomas. Minimal differences were found in the papilloma growth rates of the treated versus untreated papillomas for Treatment Groups A, B, D and E. Results from Treatment Group C are depicted in FIG. 10 and demonstrate a reduction in the size of the ISS treated papillomas compared to untreated papillomas.

### Example 12

### ISS Activity in HIV Assay

ISS activity is tested on HIV infected human peripheral blood mononuclear cell (PBMCs) in cell culture. One or more HIV virus isolates are tested with ISS-containing polynucleotides, such as SEQ ID NO:1 and appropriate controls. After infection with HIV, an ISS-containing polynucleotide is mixed with the cells and subsequent HIV production is determined through detection of p24 core antigen in the culture supernatant (which indicates amount of virus present).

Human donor PBMCs are isolated using methods well-known in the art. If the cells are frozen, sufficient numbers of cells for the assay (1×10⁷ cells/assay plate) are thawed 24 hours prior to infection. The cells are stimulated with phytohemagglutinin-P (PHAP) immediately before use. The PBMCs are collected by centrifugation and resuspended in 500 μl of HIV virus at a multiplicity of infection of 0.001 in complete RPMI media (RPMI 1640+10% FBS+20 μg/ml Gentamicin) containing polybrene at a final concentration of 2 μg/ml. The cells+virus are incubated for 4-6 hours at 37° C. Following incubation, virus is removed from the cells by centrifugation, the cells are resuspended in complete RPMI media plus 10% IL-2 and plated into a 96-well plate (150 μl/well) containing 50 μl of appropriate ISS test or control solutions. The final concentration of cells on each plate is 1×10⁵/well. The plate is covered and incubated at 37° C., 5% CO₂ for 4 days.

All test and control solutions are assayed in triplicate. Five-fold serial dilutions are made for each test ISS and control solution. Each assay plate contains a row of uninfected cells and a row of infected cells, each without test or control solutions, as positive and negative controls.

The amount of HIV produced in each well is determined using an ELISA system for the detection of HIV-1 p24 core antigen with kit from Organon Teknika (Vironostika). The assay has a linear range of 5-80 pg/ml. The amount of p24 produced in the virus control wells is above this range. Therefore, a dilution series of supernatant from these wells is prepared and tested to determine the dilution factor for the plate that will bring it in the linear range of the assay. The absorbance readings obtained from the plate is used to determine the effective concentration of the ISS solutions tested. The readings obtained from the cell control are subtracted from the data wells as background and the readings from the virus control are considered 100% infection or 0% inhibition. Accordingly, a dilution factor for the plate that gives at least a 1.5 OD difference in absorbance between the cell control and virus control is selected.

Following the 4 day incubation, p24 in positive control wells (i.e., infected cells without test or control solutions) is determined as follows. 5 μl of cell supernatant from each control well is removed. 5-fold dilutions in PBS are performed such that dilutions of 1:5, 1:25, 1:125, 1:625, 1:3125, 1:15625 are achieved. 100 μl of each dilution are assayed following the procedures described in the Vironostika test kit. Dilutions of kit control are included on the plate to obtain a calibration curve. The 96-well test plate containing the cells and remaining cell supernatants is frozen until the positive control wells are assayed.

The absorbance values vs. dilution factor for each virus control tested are plotted. A dilution factor is chosen from this curve that will result in a OD reading of approximately 1.0. The cell supernatants on the 96-well test plate are then diluted according to the chosen dilution factor and the amount of p24 is determined.

The level of p24 in the PBMC culture supernatant indicates the amount of HIV produced in the presence of the ISS or control solutions.

The present invention has been detailed both by direct description and by example. Equivalents and modifications of the present invention will be apparent to those skilled in the art, and are encompassed within the scope of the invention.

1: A method of reducing severity of a symptom of virus infection in an individual infected with a virus, comprising administering a composition comprising a polynucleotide comprising an immunostimulatory sequence (ISS) to said individual, wherein the ISS comprises the sequence 5′-C, G, pyrimidine, pyrimidine, C, G-3′, wherein an antigen of the virus is not administered in conjunction with administration of said composition, and wherein said composition is administered in an amount sufficient to reduce severity of a symptom of virus infection.

2: The method of claim 1, wherein the ISS comprises the sequence
5′-purine, purine, C, G, pyrimidine, pyrimidine, C, G-3′.

3: The method of claim 2, wherein the ISS comprises a sequence selected from the group consisting of 5′-AACGTTCG-3′, and 5′-GACGTTCG-3′.

4: The method of claim 1, wherein the ISS comprises the sequence

| | |
|---|---|
| 5′-TGACTGTGAACGTTCGAGATGA-3′. | (SEQ ID NO: 1) |

5: The method of claim 1, wherein the individual is a mammal.

6: The method of claim 1, wherein administration is at the site of infection.

7: A kit for use in ameliorating or preventing a symptom of virus infection in an individual infected with, exposed to or at risk of being exposed to a virus, comprising
a composition comprising a polynucleotide comprising an immunostimulatory sequence (ISS), wherein the ISS comprises the sequence 5′-C, G, pyrimidine, pyrimidine, C, G-3′ and wherein the kit does not comprise an antigen of the virus; and
instructions for administration of said composition to an individual infected with, exposed to or at risk of being exposed to the virus.

8: The kit of claim 7, wherein the ISS comprises the sequence
5′-purine, purine, C, G, pyrimidine, pyrimidine, C, G-3′.

9: The kit of claim 8, wherein the ISS comprises a sequence selected from the group consisting of 5′-AACGTTCG-3′, and 5′-GACGTTCG-3′.

10: The kit of claim 7, wherein the ISS comprises the sequence

| | |
|---|---|
| 5′-TGACTGTGAACGTTCGAGATGA-3′. | (SEQ ID NO: 1) |

11: A method of reducing recurrence of a symptom of virus infection in an individual infected with a virus, comprising administering a composition comprising a polynucleotide comprising an immunostimulatory sequence (ISS) to said individual, wherein the ISS comprises the sequence 5′-C, G, pyrimidine, pyrimidine, C, G-3′, wherein an antigen of the virus is not administered in conjunction with administration of said composition, and wherein said composition is administered in an amount sufficient to reduce recurrence of a symptom of virus infection.
